(19) United States
(12) Patent Application Publication
(10) Pub. No.: US 2026/0325624 A1
(43) Pub. Date: Oct. 22, 2026

(54) SARBECOVIRUS NANOVACCINES

(71) Applicant: IOWA STATE UNIVERSITY RESEARCH FOUNDATION, INC., Ames, IA (US)

(72) Inventors: Balaji NARASIMHAN, Ames, IA (US); Kathleen A. ROSS, Ames, IA (US); Sean M. KELLY, Ames, IA (US); Surya K. MALLAPRAGADA, Ames, IA (US); Michael J. WANNEMUEHLER, Ames, IA (US)

(21) Appl. No.: 19/484,552

(22) PCT Filed: May 17, 2024
(86) PCT No.: PCT/US2024/030081

§ 371 Date: Nov. 14, 2025

Related U.S. Application Data
(60) Provisional application No. 63/503,110, filed on May 18, 2023.

(51) Int. Cl. A61K 39/215; A61K 9/50; A61K 39/00; A61K 39/39; A61P 37/04

(52) U.S. Cl. CPC A61K 39/215; A61K 9/5031; A61K 39/39; A61P 37/04; A61K 2039/55505; A61K 2039/55516; A61K 2039/55555; A61K 2039/55561; A61K 2039/55577; A61K 2039/55583

(57) ABSTRACT

An immunogenic composition comprising one or more polymers, wherein the polymers comprise one or more monomers of 1,8-bis(p-carboxyphenoxy)-3,6-dioxaoctane (CPTEG), 1,6-bis(p-carboxyphenoxy) hexane (CPH), sebacic anhydride (SA); a SA homopolymer; a CPTEG:CPH copolymer; a CPTEG:SA copolymer; a CPH:SA copolymer; a CPH homopolymer; or a polyester, at least one immunogenic protein of a sarbecovirus; and at least one adjuvant, wherein a first amount of the immunogenic protein and a first amount of the adjuvant are encapsulated within the biodegradable particles, and methods of using the same.

### RELATED APPLICATIONS

This application claims priority under 35 U.S.C. § 119 (e) to U.S. Provisional Patent Application No. 63/503,110, filed May 18, 2023, which is incorporated herein by reference.

### BACKGROUND OF THE INVENTION

The emergence of severe acute respiratory syndrome coronavirus (SARS-CoV)-2 in late 2019 resulted in the COVID-19 pandemic, a public health crisis causing more than 750 million infections and 6.8 million deaths worldwide. While SARS-CoV-2 has fairly high sequence homology to the beta-coronaviruses SARS-CoV and Middle East respiratory syndrome (MERS)-CoV, SARS-CoV-2 has largely overshadowed both due to the high transmissibility of the virus. Due to its similar genetic sequence homology to SARS-CoV, many aspects of viral pathogenicity are similar. This includes viral entry via the spike(S) homotrimer viral surface protein, which binds to the angiotensin-converting enzyme 2 (ACE2) receptor to initiate host cell binding and is subsequently cleaved by furin into S1 and S2 components; additional antigenic targets that play a role in viral virulence include the envelope (E), membrane (M), and nucleocapsid (N) proteins (Jackson et al., Nat. Rev. Mol. Cell. Biol. 23 (1) (2022) 3-20).

ACE2 receptor expression in humans is abundantly present in the oral, nasal, nasopharynx, and lung epithelium. Due to this, the predominant mode of human-to-human transmission is believed to be through inhaling aerosols or droplets containing the virus. ACE2 receptor expression is also found in other tissues such as the spleen, liver, kidneys, lymph nodes, and stomach. Hence, later stages of viral infection can become systemic, leading to more severe disease in unvaccinated and immunocompromised individuals.

With the rapid progression of the COVID-19 pandemic, the initial response of the scientific community was to develop vaccines against SARS-CoV-2 to slow the spread of the virus and reduce disease severity. These efforts focused on inclusion of S antigen as the sole vaccine target, based on the observation that antibodies that bind to the receptor binding domain (RBD) of S antigen can block (i.e., neutralize) initial stages of viral infection. This proved effective, as neutralizing antibody has been shown to correlate with immune protection from SARS-CoV-2. However, these neutralizing antibody responses wane rapidly, and booster immunizations are required to maintain protection. Additionally, given the rapid replication cycle and worldwide spread of SARS-CoV-2, several viral variants of concern have emerged with observed mutations in the S protein, resulting in a change in viral fitness and ability to evade neutralizing antibodies. These resulting “breakthrough” infections highlight the need for improved vaccines that can induce robust and durable neutralizing antibodies, as well as broad humoral and cellular immunity to protect against new and unforeseen viral variants.

Accordingly, a need exists for an easily administered immunogenic sarbecovirus composition capable of producing a humoral and cell-mediated immunogenic response that is both systemic and tissue-specific. The present invention satisfies this need.

### SUMMARY OF THE INVENTION

Polyanhydride particle-based vaccines (i.e., nanovaccines) represent a promising next-generation vaccine adjuvant and delivery platform for SARS-CoV-2 vaccines. These hydrophobic polymers are capable of encapsulating vaccine antigens and additional co-adjuvants into a solid polymer matrix. The polymer matrix, which undergoes surface erosion and degradation into safe and biocompatible dicarboxylic acid monomers, enables tunable sustained release of encapsulated payloads. Sustained release from polyanhydride particles has been shown to dramatically enhance the immunogenicity of antigenic payloads, which allows for dose sparing. The physicochemical properties of polyanhydride particles confer them with inherent adjuvanticity, which has been shown to enhance both humoral and cell-mediated immune responses, and boost protection against a number of viral and bacterial respiratory pathogens such as influenza A virus, respiratory syncytial virus, Yersinia pestis (causative agent of pneumonic plague), and Streptococcus pneumoniae.

Pentablock copolymer (PBC) micelles represent another promising class of adjuvant and delivery vehicles. These micelles, composed of Pluronic® F127 and poly(diethylaminoethylmethacrylate) (PDEAEM) blocks, have been shown to induce rapid development of antibody responses via antigen presentation that crosslinks B cell receptors and induces B cell activation. Additionally, PBC micelles have been shown to facilitate cytosolic delivery of immunogens to antigen presenting cells (APCs), which may be important for the generation of cell-mediated immunity (i.e., endogenous pathway of antigen processing). As evidence of this, a vaccine formulation composed of PBC micelles and polyanhydride particles was found to synergistically induce protection against influenza A virus infection in mice.

As evidence for the rational design of combination nanovaccines that include both the polyanhydride nanoparticles and pentablock copolymers, we have previously shown that the combination of PBC micelles and polyanhydride nanoparticles was found to synergistically reduce viral load in the lungs of animals and provide protection against influenza A virus infection in mice, compared to administering the polyanhydride nanoparticles or PBC micelles formulations alone. This approach highlights the ability of combination nanovaccines to enhance protective immunity to respiratory pathogens by exploiting the unique adjuvant capability of each component in the formulation.

A class of adjuvants that shows promise for the development of vaccines against respiratory pathogens is cyclic dinucleotides (CDN). These small molecule agonists are recognized as microbial associated molecular patterns (MAMPs) by the pattern recognition receptor Stimulator of INterferon Genes (STING), resulting in cellular activation via the NFκB and TBK1/IRF3 immune signaling pathways and activation of antiviral activity. Of note, cyclic di-guanosine monophosphate (c-di-GMP) is the most studied CDN to date which possesses potent adjuvant activity for subunit antigens by inducing cell-mediated immunity and durable, high titer antibody responses following immunization. Vaccination with antigens adjuvanted with CdG have been shown to protect against numerous respiratory pathogens including Y. pestis and influenza A virus and induce anti-tumor responses.

This disclosure describes a versatile, plug-and-play platform technology that was used to design multiple nanovaccine formulations containing S and N antigens derived from wild-type SARS-CoV-2 (Wuhan-Hu-1) and evaluated their ability to induce durable, systemic antibody responses that have the ability to bind to RBDs of multiple SARS-CoV-2 variants. We also show the ability of SARS-CoV-2 nanovaccines to induce antigen-specific T cell responses. Lastly, we demonstrate that the nanovaccines can protect animals against lethal challenge with live SARS-CoV-2 virus.

Accordingly, in one aspect, an immunogenic composition comprises biodegradable particles comprising one or more polymers, wherein the polymers comprise one or more monomers of 1,8-bis(p-carboxyphenoxy)-3,6-dioxaoctane (CPTEG), 1,6-bis(p-carboxyphenoxy) hexane (CPH), sebacic anhydride (SA), a SA homopolymer, a CPTEG:CPH copolymer, a CPTEG:SA copolymer, a CPH:SA copolymer, a CPH homopolymer, or a polyester copolymer; at least one immunogenic protein of a sarbecovirus; and at least one adjuvant; wherein a first amount of the immunogenic protein and a first amount of the adjuvant are encapsulated within the biodegradable particles.

In another aspect, an immunogenic composition may further comprise pentablock copolymer micelles, wherein a second amount of the immunogenic protein, a second amount of the adjuvant, or both are entrapped within one or more of the pentablock copolymer micelles.

In another aspect, the particles comprise the CPTEG:CPH copolymer in a molar ratio of about 20:80; CPTEG:SA copolymer in a molar ratio of 10:90; or CPH:SA copolymer in a molar ratio of 20:80.

In another aspect, the pentablock copolymer micelle comprise poly((diethylamino)-ethyl methacrylate) (PDEAEM), a polyolefin elastomer (POE), and a polyolefin plastomer (POP) block. In some aspects, the pentablock copolymer micelle comprise PDEAEM and a POE block, wherein the POE is poloxamer 407.

Exemplary adjuvants include a Stimulator of Interferon Genes (STING) activating adjuvant, a Toll-like receptor (TLR) agonist, a nucleotide oligomerization domain (NOD)-like receptor (NLR) agonist, a RIG-like receptor (RLR), a liposome, an aluminum salt, a mineral salt, an oil emulsion, a polymer, a polysaccharide, a saponin, or a CpG polynucleotide.

In some aspects, the composition comprises about 0.5 milligrams to about 100 milligrams of the pentablock copolymer micelles. The immunogenic protein and the adjuvant may be present in the pentablock copolymer micelles in an amount of about 0.1 micrograms to about 500 micrograms each.

The composition may comprise about 5 micrograms to about 1000 milligrams of the biodegradable particles. The immunogenic protein and the adjuvant may be present in the biodegradable particles in an amount of about 0.1 micrograms to about 500 micrograms each.

In some aspects, the sarbecovirus is severe acute respiratory syndrome coronavirus (SARS-CoV), severe acute respiratory syndrome coronavirus 2 (SARS-CoV-2), Middle East respiratory syndrome coronavirus (MERS-CoV), or a variant thereof.

In some aspects, the immunogenic protein comprises one or more of a spike protein (S protein), a nucleocapsid protein (N protein), an envelope protein (E protein), and a membrane protein (M protein); or the immunogenic protein comprises a sarbecovirus spike protein (S protein) and a sarbecovirus nucleocapsid protein (N protein).

In one aspect, an immunogenic composition may comprise biodegradable polyanhydride particles comprising 1,8-bis(p-carboxyphenoxy)-3,6-dioxaoctane (CPTEG) and 1,6-bis(p-carboxyphenoxy) hexane (CPH) copolymers in a molar ratio of about 20:80; pentablock copolymer micelles comprising poloxamer 407 and poly((diethylamino)-ethyl methacrylate) (PDEAEM); a spike protein (S protein) and a nucleocapsid protein (N protein) of SARS-CoV-2 virus; and an adjuvant comprising cyclic di-guanosine monophosphate (c-di-GMP); wherein a first amount of the S protein, a first amount of the N protein, and a first amount of the adjuvant are encapsulated within the biodegradable particles; and a second amount of the S protein, a second amount of the N protein, and a second amount of the adjuvant are entrapped within the pentablock copolymer micelles. The first amount of the S protein, the first amount of the N protein, the second amount of the S protein, and the second amount of the N protein, are present in an amount of about 0.1 micrograms to about 500 micrograms each; and the first amount of the adjuvant and the second amount of the adjuvant are present in an amount of about 0.1 micrograms to about 500 micrograms each.

The disclosure also provides for methods of inducing an immune response against a sarbecovirus in a subject comprising administering to the subject an effective amount of the immunogenic composition as described herein, thereby inducing an immune response. In some aspects, the immune response comprises both a local immune response and systemic protective immune response to sarbecovirus. Preferably, an immunogenic composition is administered intranasally and/or parenterally.

The present invention and its attributes and advantages will be further understood and appreciated with reference to the detailed description below of presently contemplated embodiments, taken in conjunction with the accompanying drawings.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-B. SARS-CoV-2 nanovaccine induced rapid and long-lived antibody titers. (A) Total anti-S IgG antibody titers through 62 weeks after primary immunization of female C57BL/6 mice (6-8 wk) administered a SARS-CoV-2 nanovaccine (prime-boost, 21 days apart). n=4. (B) Total anti-S IgG antibody titers through 30 weeks after primary immunization of female C57BL/6 mice (6-8 wk) administered a SARS-CoV-2 nanovaccine co-encapsulating S, N, and CdG (prime-boost, 21 days apart). n=3-4. Error bars indicate standard error of mean. LOD=limit of detection. * indicates significance in comparison to naïve at specified time point as determined by Mann-Whitney test, p≤0.05.

FIG. 2A-D. SARS-CoV-2 nanovaccine induced S- and N-specific CD4⁺ and CD8⁺ T cell responses. Percentage of CFSE^{low} CD4⁺ and CD8⁺ T cells collected from (A) axillary/brachial lymph nodes and (C) spleen of female C57BL/6 mice 45 days after primary immunization with SARS-CoV-2 nanovaccine co-encapsulating S, N, and CdG (prime-boost, 21 days apart). Cells were plated at 5×10⁵ cells/well and restimulated with S and N antigen (25 μg/mL of each antigen) for five days. Error bars indicate standard error of mean. Asterisks indicate significance in comparison to cells from naïve animals as determined by Mann-Whitney test. Cytokine response in cell supernatants from (B) axillary/brachial lymph nodes and (D) spleen after restimulation. n=7-8 mice per treatment group. Error bars indicate standard error of mean. Asterisks indicate significance in comparison to cells from naïve animals as determined by Mann-Whitney test (****p≤0.0001, ***p≤0.001, **p≤0.01, *p≤0.05). ns=not significant.

FIG. 3A-B. 2× antigen dose SARS-CoV-2 nanovaccine induced serum neutralizing antibody. (A) Optical density (OD) of systemic serum anti-S IgG antibody response 35 days after primary immunization in female K18-hACE2 mice (6-8 wk) administered a 1× or 2× antigen dose nanovaccine co-encapsulating S, N, and CdG (prime-boost, 21 days apart). Error bars indicate standard error of mean. ⁺ indicates significant difference between 1× and 2× antigen. ^{#} indicates significant difference of 1× antigen in comparison to naïve. * indicates significant difference of 2× antigen in comparison to naïve determined via two-way ANOVA with Tukey's multiple comparisons. p≤0.05. (B) Percent inhibition of RBD binding by serum antibody (i.e., surrogate measure of viral neutralization) 35 days post-immunization. n=8 mice per treatment group. Error bars indicate standard error of mean. * indicates significant difference between treatments determined by a one-way ANOVA with Tukey's multiple comparisons. p≤0.05. Controls provided by the manufacturer and used according to instructions.

FIG. 4A-D. Optimized SARS-CoV-2 nanovaccine provides protection in mice. (A) Experiment schematic, (B) weight loss kinetics, (C) survival, and (D) viral load of female K18-hACE2 mice challenged with 2.5×10⁴ PFU at 42 days after primary immunization with 1× or 2× antigen dose nanovaccine co-encapsulating S, N, and CdG (prime-boost, 21 days apart). n=14 for weight loss & survival. n=4 for viral load. Error bars indicate standard error of mean. * indicates significance difference determined by two-way ANOVA with Tukey's multiple comparisons (B), log-rank Mantel Cox curve comparison (C), or one-way ANOVA with Tukey's multiple comparisons (D), p≤0.05.

FIG. 5A-B. Representative histopathology of lungs from mice after SARS-CoV-2 viral challenge. K18-hACE2 mice receiving a prime-boost regimen of the 1× or 2× antigen dose nanovaccine were challenged with 2.5×10⁴ PFU of SARS-CoV-2. At five days post-challenge, four mice per group were euthanized and lung tissue was fixed in 10% buffered formalin and sectioned. (A) Tissue sections were stained for histopathology with hematoxylin and eosin (H&E). Scale bars=500 μm (20× magnification). Inset scale bars=100 μm (200× magnification). (B) Separate tissue sections were stained for immunohistochemical analysis using H&E and either anti-CD4, anti-CD8a, or anti-CD20. Scale bars=50 μm (200× magnification). All tissue evaluations were performed by a board-certified veterinary pathologist.

FIG. 6A-D. Prime-boost administration of SARS-CoV-2 nanovaccines generated cross-reactive antibody responses. Optical density of anti-S RBD IgG antibody response against (A) wild-type RBD (Wuhan-Hu-1), (B) Beta variant (B.1.351) RBD, (C) B.1.617 RBD, or (D) Omicron variant (B.1.1.529) RBD in sera of female K18-hACE2 mice (n=14) collected 35 days after primary immunization with the 1× or 2× antigen dose nanovaccine co-encapsulating S, N, and CdG (prime-boost, 21 days apart). Error bars indicate standard error of mean. ⁺ indicates significant difference between 1× and 2× antigen dose nanovaccines. ^{#} indicates significant difference of 1× antigen dose nanovaccine in comparison to naïve. * indicates significant difference of 2× antigen dose nanovaccine in comparison to naïve determined via two-way ANOVA with Tukey's multiple comparisons, p≤0.05. Triangle=Naïve, closed circle=1× antigen nanovaccine, square=2× antigen nanovaccine.

FIG. 7A-D. Representative scanning electron photomicrographs and particle size distribution for 1× Antigen Nanovax (A) and 2× Antigen Nanovax (B). Representative release kinetics of total protein from polyanhydride particles encapsulating S and N antigens and a model adjuvant (CpG ODN 1668) (C). SDS-PAGE demonstrating stability of S after encapsulation and release from polyanhydride nanoparticles (Released Spike) in comparison to a fresh, unencapsulated control (Fresh Spike) (D).

FIG. 8. Representative flow cytometric gating strategy for CFSE^{low} CD4⁺ and CD8⁺ T cells from axillary/brachial lymph nodes collected 45 days post-immunization and restimulated with 25 μg/mL each of S and N antigens for five days.

FIG. 9. Histopathology of lungs from mice after lethal SARS-CoV-2 viral challenge. K18-hACE2 mice receiving a prime-boost regimen of the 1× or 2× antigen dose nanovaccine were challenged with 2.5×10⁴ PFU of SARS-CoV-2. At five days post-challenge, four mice per group were euthanized and lung tissue was fixed in 10% buffered formalin, section, and stained with hematoxylin and eosin (H&E) before evaluation by a board-certified veterinary pathologist. While all lung tissue sections depicted mild interstitial pneumonia regardless of treatment, those of vaccinated mice presented with more pervasive perivascular inflammatory cells composed of lymphocytes, plasma cells, and macrophages. Scale bars=500 μm (20× magnification).

### DETAILED DESCRIPTION OF THE INVENTION

The invention provides for immunogenic compositions and methods of use that generally include a biodegradable polyanhydride particle comprising one or more sarbecovirus immunogenic proteins and an adjuvant each encapsulated within the interior of the particle. In other embodiments, certain compositions also may comprise one or more sarbecovirus immunogenic proteins and/or adjuvants entrapped within a pentablock copolymer micelle.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R.J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, PCR amplification, cell culturing, etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

An “effective amount” refers to an amount effective to kill a cell, inhibit a cell from growing or dividing, treat a disease, disorder, and/or condition, or to bring about a recited effect. For example, an effective amount can be an amount effective to reduce the progression or severity of the condition or symptoms being treated. Determination of an effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping, or reversing the progression or severity of the condition or symptoms being treated. As such, the term “treatment” can include medical, therapeutic, and/or prophylactic administration and immunization and/or vaccination, as appropriate.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

As used herein, “pharmaceutically acceptable” carrier or excipient includes any and all solvents, dispersion media, coatings, antibacterial and antifungal agents, isotonic and absorption/resorption delaying agents, and the like that are physiologically compatible. Preferably, the carrier is suitable for injection or infusion. Pharmaceutically acceptable carriers include sterile aqueous solutions or dispersions and sterile powders for the preparation of sterile injectable solutions or dispersion. The use of such media and agents for pharmaceutically active substances is known in the art. In addition to water, the carrier can be, for example, an isotonic buffered saline solution.

As used herein, the terms “purified” and “isolated” when used in the context of a polypeptide (including an antibody) that is obtained from a natural source, e.g., cells, refers to a polypeptide which is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. Thus, a polypeptide that is isolated includes preparations of a polypeptide having less than about 30%, 20%, 10%), 5%, 2%, or 1%> (by dry weight) of cellular materials and/or contaminating materials. As used herein, the terms “purified” and “isolated” when used in the context of a polypeptide (including an antibody) that is chemically synthesized refers to a polypeptide which is substantially free of chemical precursors or other chemicals which are involved in the syntheses of the polypeptide.

As used herein, terms “subject” or “patient” are used interchangeably to refer to an animal (e.g., birds, reptiles, and mammals). In a specific embodiment, a subject is a bird. In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In another embodiment, a subject is a human.

As used herein, “entrapped” refers to the incorporation or partial incorporation of an immunogenic protein and/or adjuvant into and/or onto the matrix of a polyanhydride particle or the pentablock copolymer micelle. The properties of the polymers may be tailored through the selection of various monomers having the appropriate properties for entrapment and delivery of the immunogenic payload, such as, for example, a vaccine.

As used herein, “encapsulate” or “encapsulated” refers to the incorporation of an immunogenic protein or adjuvant within the polyanhydride particle or the pentablock copolymer micelle.

In some embodiments, an amount of a component of a composition is expressed in weight/weight, weight/volume, volume/volume, or weight percentage as appropriate. Preferably, an amount of a component of a composition is expressed as w/w (weight of the compound/total weight of the composition).

### Embodiments of the Invention

The present invention generally is directed to certain immunogenic compositions that may include one or more biodegradable polymer particles containing an effective amount of at least one sarbecovirus immunogenic protein, and in particular, SARS-CoV-2 immunogenic proteins, one or more adjuvants encapsulated within the particle, and optionally, pentablock copolymer micelles containing an effective amount of at least one sarbecovirus immunogenic protein and one or more adjuvants entrapped within the pentablock copolymer micelles. Immunogenic compositions may be used to illicit an immune response and the formation of local tissue resident T and B memory cells.

The polymer particles of the immunogenic composition can be any one or more of the following biodegradable polymers. In various embodiments, the biodegradable polymer particles comprise polyanhydride homopolymers and/or copolymers of 1,ω-bis(p-carboxyphenoxy)alkanes and 1,ω-dicarboxylic alkanes such as 1,6-bis(p-carboxyphenoxy) hexane wherein every third carbon of the alkane moiety is optionally replaced with an oxygen to form one or more ethylene glycol moieties. Other polyanhydride copolymers preferably include poly(bis-(1,ω-carboxyphenoxy) (C₂-C₁₂)alkane-co-(C₅-C₂₀)bis-alkanoic acids). The substituents on the phenoxy moiety may be orientated ortho, meta, or para to each other, and are typically in a para relationship. The alkane moiety of the bis-(carboxyphenoxy)alkane may be a (C₂-C₁₂)alkane. In more specific embodiments, the alkane moiety may be a (C₄-C₈)alkane, and more specifically a (C₆)alkane. The alkanoic diacids used for the biodegradable polymers may be a (C₅-C₂₀)alkane bis-carboxylic acid. Specifically, the bis-carboxylic acid may be a (C₆-C₁₆)alkane bis-carboxylic acid, a (C₈-C₁₂)alkane bis-carboxylic acid, or more specifically a (C₁₀)alkane bis-carboxylic acid (i.e., sebacic acid). Optionally, the alkane and aryl moieties of the polyanhydride copolymers may be substituted in a manner that increases or decreases hydrophobicity of the particles.

A general formula for one embodiment of the copolymers (which covers CPTEG:CPH) is:

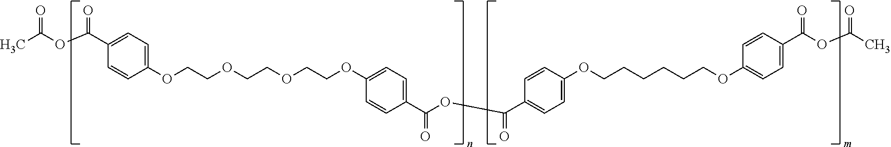

A general formula for one embodiment of the copolymers (which covers CPH:SA) is:

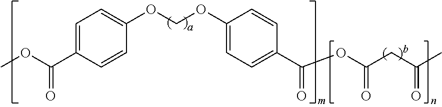

wherein m and n represent the number of repeating units of each monomer, for example, such that the polymers have molecular weights of about 4,000 to about 55,000. The variables “m” and “n” are not less than one and are typically greater than ten. The variable “a” may be about 2-12 and the variable “b” may be about 2-20. In certain specific embodiments, polyanhydride homopolymers may also be used, in which case either m or n would be zero. In some embodiments, the polymer is a copolymer of 1,8-bis(p-carboxyphenoxy)-3,6-dioxaoctane (CPTEG) and 1,6-bis(p-carboxyphenoxy) hexane (CPH) (abbreviated as CPTEG:CPH). In another embodiment, the polymer is a copolymer of CPTEG and sebacic anhydride (SA) (abbreviated as CPTEG:SA). In another embodiment, the polymer is a copolymer of CPH and SA (abbreviated as CPH:SA). In some embodiments, the CPTEG:SA copolymer comprises a molar ratio of 10:90; or the CPH:SA copolymer comprises a molar ratio of 20:80. The molecular weight range of the CPTEG:CPH copolymer is generally about 5,000 to about 10,000 g/mol, the molecular weight range of the CPTEG:SA copolymer is generally about 10,000 to about 30,000 g/mol, and the molecular weight range of the CPH:SA copolymer is generally about 10,000 to about 30,000 g/mol.

In specific embodiments, a particles may comprise SA or a SA homopolymer, a CPTEG:CPH copolymer, a CPTEG:SA copolymer, a CPH:SA copolymer, or a CPH homopolymer. In some embodiments, the biodegradable particles comprise a CPTEG:CPH copolymer in a molar ratio of about 20:80, the CPTEG:SA copolymer in a molar ratio of 10:90, or the CPH:SA copolymer in a molar ratio of 20:80. In some embodiments, the polymer may be, for example, a polyester such as poly(lactic acid-co-glycolic Acid) (PLGA), polyglycolic acid, polylactic acid, poly(l-caprolactone), or another polyester such as poly(ether ester amide).

The polymers and copolymers may be synthesized through, for example, melt polycondensation from acetylated prepolymers using the techniques described by Kipper et al. ((2002) Biomaterials 23, 4405-4412), Torres et al. ((2006) J. Biomed. Mater. Res. A 76, 102-110), U.S. Pat. No. 7,858,093 (Kipper et al.), or U.S. Pat. No. 8,449,916 (Bellaire and Narasimhan), which describe examples of useful copolymers that can be used for the particles described herein, and which publications and patents are incorporated herein by reference. The polyanhydrides copolymers also may be prepared by microwave polymerization as described by Vogel et al. (2004) Macromol. Rapid Comm., 25, 330-333). Other techniques known to those of skill in the art also may be used to prepare the copolymers. The prepared polyanhydride copolymers can have molecular weights of about 4,000 to about 55,000, specifically about 5,000 to about 30,000, and more specifically about 5,000 to about 20,000, or other ranges as described herein.

Any suitable and effective ratio of monomers may be used in the synthesis of the polyanhydride copolymers. For example, the carboxyphenoxyalkane (CPA) to alkanoic diacid (AD) ratio may be about 1:1 to about 1:10. Certain embodiments include monomer ratios of about 1:1, about 1:1.5, about 1:2, about 1:3, about 1:4, about 1:5, and about 1:9.

In some embodiments, the particles are bioerodible or biodegradable. For example, some particles may degrade through surface-erosion due to the in vivo hydrolysis of anhydride linkages at the surface of the particle that may result in the controlled release of immunogen(s) to a subject. Surface-erodible biomaterials useful for the delivery of immunogens by the techniques disclosed herein are described by, for example, Narasimhan and Kipper, ((2004) Adv. Chem. Eng., 29, 169-218). Microstructural characterization of polyanhydride blends for controlled drug delivery are described by, for example, Mallapragada et al., Biomaterials for Drug Delivery and Tissue Engineering, Eds. (2001) Mater. Res. Soc. Symp. Proc. 662, NN4.2.1-4.2.5). Typically, particles of the invention are substantially spherical with an average diameter of about 150 nm to about 5 μm and a Polydispersity Index of about 0.1 to about 0.2.

Biodegradable or bioerodible polymers may be used to encapsulate one or more immunogenic proteins or antigen, and, optionally, one or more adjuvants for delivery to a subject. As used herein, the term “immunogenic protein” refers to a molecule with one or more epitopes that stimulate a host's immune system to make a secretory, humoral and/or cellular antigen-specific response against one or more strains of sarbecovirus in a vertebrate. The term is also used interchangeably with “immunogen”. By way of example, embodiments may include one or more specific immunogenic proteins that may be a complete protein, portions of a protein, a peptide, fusion proteins, glycosylated proteins, and combinations thereof. In certain preferred embodiments, the immunogenic protein is a full-length sarbecovirus protein.

Particles of the present inventions may include any combination of immunogenic proteins disclosed herein. Preferred embodiments may include an immunogenic protein of a sarbecovirus. As used herein, “sarbecovirus” refers to a subgenus of genus betacoronavirus, family Coronaviridae within lineage B. Exemplary sarbecoviruses include, but are not limited to, severe acute respiratory syndrome coronavirus 1 (SARS-CoV-1), SARS-CoV-2, Middle East Respiratory Syndrome-related coronavirus (MERS-CoV), Bat SARS-like coronavirus WIV1 (WIV1), RatG13, Pangolin-Guanxi-2017 (PANG/GX), Pangolin-Guangdon-2019 (PANG/GD).

Examples of SARS-CoV-2 variants, include, for example, N501Y, Y453F, N439K, K417V, E484K, N501Y-K417N-E484K, B.E1.7, B.1.351, B.1.429, P.1, B.1.1.7, B.1.1.222, B.1.617.2, B.1.1.529, C.37, AY.1, AY.2, a California variant, a Brazilian variant, and a Swiss variant. Other sarbecoviruses are described in International Patent Publication No. WO 2022/204202.

In some embodiments, an immunogenic protein may include, but is not limited to, a spike protein (S protein), a nucleocapsid protein (N protein), an envelope protein (E protein), and a membrane protein (M protein) of a sarbecovirus. In some embodiments, the one or more immunogenic proteins comprises a spike protein (S protein) and a nucleocapsid protein (N protein) of a sarbecovirus. In other embodiments, the immunogenic protein may be a portion or sub peptide of, for example, a spike protein (S protein), a nucleocapsid protein (N protein), an envelope protein (E protein), and a membrane protein (M protein) of a sarbecovirus.

In other embodiments, the immunogenic proteins are derived from SARS-CoV-2 or SARS-CoV-2 variants. These immunogenic proteins include, but are not limited to, a spike protein (S protein), a nucleocapsid protein (N protein), an envelope protein (E protein), and a membrane protein (M protein). In some embodiments, the one or more immunogenic proteins from SARS-CoV-2 or SARS-CoV-2 variant comprises a spike protein (S protein) and a nucleocapsid protein (N protein). In other embodiments, the immunogenic protein may be a portion or sub peptide of, for example, a spike protein (S protein), a nucleocapsid protein (N protein), an envelope protein (E protein), and a membrane protein (M protein). (See U.S. Pat. No. 11,684,669 to Meinke et al.; U.S. Pat. No. 11,931,410 to Hu et al.; U.S. Publication No. 2023/0263,881 to Ascione et al.; and 2023/0070,886 to Smith et al.).

In some embodiments, the Spike protein is from the ancestral Wuhan-Hu-1 strain of SARS-CoV-2 (Accession No. YP_009724390.1):

(SEQ ID NO: 1)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRENGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT.

In some embodiments, the nucleocapsid protein is from the ancestral strain of SARS-CoV-2, (Accession No. YP_009724397.2):

(SEQ ID NO: 2)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA.

In some embodiments, membrane protein is from the ancestral strain of SARS-CoV-2 (Accession No. YP_009724393.1):

(SEQ ID NO: 3)
MADSNGTITVEELKKLLEQWNLVIGFLFLTWICLLQFAYANRNRFLYIIK
LIFLWLLWPVTLACFVLAAVYRINWITGGIAIAMACLVGLMWLSYFIASF
RLFARTRSMWSFNPETNILLNVPLHGTILTRPLLESELVIGAVILRGHLR
IAGHHLGRCDIKDLPKEITVATSRTLSYYKLGASQRVAGDSGFAAYSRYR
IGNYKLNTDHSSSSDNIALLVQ.

In some embodiments, envelope protein is from the ancestral strain of SARS-CoV-2 (Accession No. YP_009724392.1):

(SEQ ID NO: 4)
MYSFVSEETGTLIVNSVLLFLAFVVFLLVTLAILTALRLCAYCCNIVNVS
LVKPSFYVYSRVKNLNSSRVPDLLV.

It is contemplated that any existing or future variants of the spike, nucleocapsid, membrane, and envelope protein may be used with the invention.

Certain preferred embodiments also include one or more adjuvants. As used herein, the term “adjuvant” refers to a compound that may augment, enhance and/or boost the immune response to an immunogen. In some embodiments, the adjuvant generates an immune response to the immunogen and does not produce an allergy or other adverse reaction. Adjuvants can enhance an immune response by serving as a depot and by several mechanisms including, e.g., lymphocyte recruitment, stimulation of B and/or T cells, and stimulation of cells of the innate immune system (e.g., dendritic cells, macrophages).

Specific examples of adjuvants include, but are not limited to, aluminum salts (such as aluminum hydroxide, aluminum phosphate, and aluminum sulfate), 3 de-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Publication No. WO2007/109812 to Sutton et al.), imidazoquinoxaline compounds (see International Publication No. WO 2007/109813 to Sutton et al.) and saponins, such as QS21 (see Kensil et al., in Vaccine Design: The Subunit and Adjuvant Approach (eds. Powell & Newman, Plenum Press, NY, 1995); and U.S. Pat. No. 5,057,540 to Kensil et al.). In some embodiments, the adjuvant is Freund's adjuvant (complete or incomplete). Other adjuvants include oil in water emulsions (such as squalene or peanut oil), cholera toxin B subunit, flagellin, human papillomavirus L1 or L2 protein, herpes simplex glycoprotein D (gD), complement C4 binding protein, TLR4 ligands such as MPL, and IL-1B, lysolecithin, pluronic polyols, polyanions, dinitrophenol, iscomatrix, liposome polycation DNA particles, CpG polynucleotides, and Stimulator of Interferon Genes (STING) activating adjuvants.

Furthermore, adjuvants and specific examples of adjuvants that can be included in the particles are provided in Table 1 below.

TABLE 1
Exemplary adjuvants.

| Adjuvant Class | Examples |
|---|---|
| Liposomes | AS01 |
| | Virosomes |
| | Virus-like particles |
| Mineral Salts | Aluminum hydroxide |
| | Aluminum phosphate |
| | Calcium phosphate |
| Oil Emulsions | MF59 |
| | AS02 |
| | AS03 |
| Polymers | Polyanhydrides (CPH, CPP, SA, |
| | CPTEG) |
| | Polysulfones |
| | Polyesters (PLGA, PLA, PGA, PCL) |
| | Micelles (PDEAEM, Pluronic F127) |
| | Polyethers (PEG) |
| | Poly(vinyl alcohol) |
| | Poly(caprolactone) |
| | Poly(hydroxybutyrate) |
| | Chitosan |
| Polysaccharides | β-glucans |
| | Mannose |
| Saponins | Quillaja saponins |
| | Quill A |
| | QS-21 |
| | ISCOMs |
| STING-activating | Cyclic dinucleotides (CDNs) |
| | R,R-CDG |
| TLR Agonists | CpG Oligodeoxynucleotide |
| | Poly I:C |
| | Imiquimod/Resiquimod |
| | MPLA |
| | Flagellin |
| | AS04 |

In some embodiments, the adjuvant may include one or more of a Toll-like receptor (TLR) agonist, a nucleotide oligomerization domain (NOD)-like receptor (NLR) agonist, a RIG-like receptor (RLR), a liposome, an aluminum salt, a mineral salt, an oil emulsion, a polymer, a polysaccharide, a saponin, a CpG polynucleotide, and a Stimulator of Interferon Genes (STING) activating adjuvant.

In some embodiments, the TLR agonist comprises one or more of triacylated lipoproteins, lipoteichoic acid, peptidoglycans, Zymosan, Pam3CSK4, Diacylated lipopeptides, HSPs, HMGB1, uric acid, fibronectin, ECM proteins, double stranded RNA, poly I: C, lipopolysaccharides, β-defensin, 2, fibronectin EDA, HMGB1, saponin, tenascin C, flagellin, single stranded RNA, CpG-A, poly G10, poly G3, and unmethylated CpG DNA. Examples of TLR agonists are described in, for example, Kaczanowska et al., J Leukoc Biol. 2013 June; 93 (6): 847-863.

In some embodiments, the STING agonist cyclic dinucleotide is selected from the group consisting of cyclic di-guanosine monophosphate (c-di-GMP), cyclic di-adenosine monophosphate (c-di-AMP), or cyclic guanosine monophosphate-adenosine monophosphate (cGAMP).

It is contemplated that one or more adjuvants may be entrapped or, more preferably, encapsulated in the interior of a particle. Alternatively, compositions of the invention may include an adjuvant in pentablock copolymer micelles, but not in the interior of the particle. In some embodiments, both the particle and the pentablock copolymer micelles may include one or more adjuvants.

In some embodiments, an immunogenic composition comprises biodegradable particles comprised of one or more polymers, wherein the polymers comprise one or more monomers of 1,8-bis(p-carboxyphenoxy)-3,6-dioxaoctane (CPTEG), 1,6-bis(p-carboxyphenoxy) hexane (CPH), sebacic anhydride (SA), a SA homopolymer, a CPTEG:CPH copolymer, a CPTEG:SA copolymer, a CPH:SA copolymer, a CPH homopolymer, or a polyester copolymer; at least one immunogenic protein of a sarbecovirus; and at least one adjuvant; wherein a first amount of the immunogenic protein and a first amount of the adjuvant are encapsulated within the biodegradable particles.

In another embodiment, an immunogenic composition comprises biodegradable particles comprising one or more polymers, wherein the polymers comprise one or more monomers of 1,8-bis(p-carboxyphenoxy)-3,6-dioxaoctane (CPTEG), 1,6-bis(p-carboxyphenoxy) hexane (CPH), sebacic anhydride (SA), a SA homopolymer, a CPTEG:CPH copolymer, a CPTEG:SA copolymer, a CPH:SA copolymer, a CPH homopolymer, or a polyester copolymer; at least one immunogenic protein of a sarbecovirus; and at least one adjuvant; and pentablock copolymer micelles; wherein a first amount of the immunogenic protein and a first amount of the adjuvant are encapsulated within the biodegradable particles, and wherein a second amount of the immunogenic protein, a second amount of the adjuvant, or both are entrapped within the pentablock copolymer micelles.

In some embodiments, an immunogenic composition may include two or more populations of biodegradable particles and/or micelles comprising the same or different immunogenic proteins, polymers, and/or adjuvants.

In other embodiments, the immunogenic composition comprises a pentablock copolymer micelles, and a second amount of the one or more immunogenic proteins of sarbecovirus and a second amount of the one or more adjuvants are entrapped within the pentablock copolymer micelles.

In certain embodiments, micelles may be formed of cationic surface-active agents, anionic surface-active agents, zwitterionic surface active agents, or combinations thereof. In some embodiments, the compositions include one or more non-ionic surface-active agents. Non-ionic surface-active agents generally have a hydrophobic group and a reactive hydrogen atom, for example aliphatic alcohols, acids, amides and alkyl phenols, with alkylene oxides, especially ethylene oxide either alone or in combination with propylene oxide. Examples of nonionic surfactant compounds include, but are not limited to, polyoxyethylene glycol sorbitan alkyl esters, block copolymers of polyethylene glycol and polypropylene glycol, ethylene glycol fatty acid esters, poly(ethylene glycol) fatty acid esters, propylene glycol fatty acid esters, poly(propylene glycol) fatty acid esters, glycol fatty acid esters, trimethylolpropane fatty acid esters, pentaerythritol fatty acid esters, glucoside derivatives, glycerin alkyl ether fatty acid esters, trimethylolpropane oxyethylene alkyl ethers, fatty acid amides, alkylolamides, alkylamine oxides, lanolin and its derivatives, castor oil derivatives, hardened castor oil derivatives, sterols and its derivatives, polyoxyethylene alkyl ethers, polyoxyethylene alkyl allyl ethers, polyoxyethylene alkylamine, polyoxyethylene fatty acid amides, polyoxyethylene alkylolamides, polyoxyethylene diethanolamine fatty acid esters, polyoxyethylene trimethylolpropane fatty acid esters, polyoxyethylene alkyl ether fatty acid esters, polyoxyethylene polyoxypropylene glycols, polyoxyethylene polyoxypropylene alkyl ethers, polyoxyethylene polyoxypropylene polyhydric alcohol ethers, glycerin fatty acid esters, polyglycerin fatty acid esters, polyoxyethylene glycerin fatty acid esters, sorbitan fatty acid esters, polyoxyethylene sorbitan fatty acid esters, sucrose fatty acid esters, or combinations thereof.

Polysorbates are a class of oily liquids derived from PEG-ylated sorbitan (a derivative of sorbitol) esterified with fatty acids. Common brand names for polysorbates include TWEEN®, TWEEN®-20, TWEEN®-60 and TWEEN®-80, for example, are available from AkzoNobel (Strawinskylaan 2555 1077 ZZ, Amsterdam, the Netherlands). Exemplary polysorbates include polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate).

Examples of block copolymers of polyethylene glycol and polypropylene glycol include the poloxamers. Poloxamers are nonionic triblock copolymers composed of a central hydrophobic chain of polyoxypropylene (poly(propylene oxide)) flanked by two hydrophilic chains of polyoxyethylene (poly(ethylene oxide)). Exemplary poloxamers include, but not limited to, poloxamer 124, poloxamer 188, poloxamer 237, poloxamer 338, poloxamer 407 and the like. In a preferred embodiment, the poloxamer is poloxamer 407 (i.e., Pluronic® F-127):

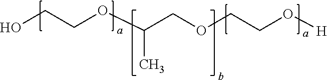

wherein poloxamer 407 has approximate block lengths of a=101 and b=56.

Certain poloxamers, such as those listed herein, are also known by the trade names PLURONIC® available from suppliers such as BASF AG (Ludwigshafen, Germany). Because the lengths of the polymer blocks can be customized, many different poloxamers exist that have slightly different properties. Further exemplary PLURONIC® poloxamers include, but are not limited to PLURONIC® 10R5, PLURONIC® 17R2, PLURONIC® 17R4, PLURONIC® 25R2, PLURONIC® 25R4, PLURONIC® 31R1, PLURONIC® F 108 Cast Solid Surfactant, PLURONIC® F 108 NF, PLURONIC® F 108 Pastille, PLURONIC® F 108 Prill, PLURONIC® F 108NF Prill Poloxamer 338, PLURONIC® F 127, PLURONIC® F 127 Prill, PLURONIC® F 127 NF, PLURONIC® F 127 NF 500 BHT Prill, PLURONIC® F 127 NF Prill Poloxamer 407, PLURONIC® F 38, PLURONIC® F 38 Pastille, PLURONIC® F 68, PLURONIC® F 68 Pastille, PLURONIC® F 68 LF Pastille, PLURONIC® F 68 NF, PLURONIC® F 68 NF Prill Poloxamer 188, PLURONIC® F 68 Prill, PLURONIC® F 68 Prill, PLURONIC® F 77, PLURONIC® F 77 Micropastille, PLURONIC® F 87, PLURONIC® F 87 NF, PLURONIC® F 87 NF Prill Poloxamer 237, PLURONIC® F 87 Prill, PLURONIC® F 88, PLURONIC® F 88 Pastille, PLURONIC® F 88 Prill, PLURONIC® F 98, PLURONIC® F 88 Prill, PLURONIC® F 98, PLURONIC® F 98 Prill, PLURONIC® L 10, PLURONIC® L 101, PLURONIC® L 121, PLURONIC® L 31, PLURONIC® L 35, PLURONIC® L 43, PLURONIC® L 44, PLURONIC® L 61, PLURONIC® L 62, PLURONIC® L 62 LF, PLURONIC® L 62D, PLURONIC® L 64, PLURONIC® L 81, PLURONIC® L 92, PLURONIC® L44 NF INH surfactant Poloxamer 124, PLURONIC® N 3, PLURONIC® P 103, PLURONIC® P 104, PLURONIC® P 105, PLURONIC® P 123 Surfactant, PLURONIC® P 65, PLURONIC® P 84, PLURONIC® P 85, or combinations thereof. In certain preferred embodiments, the poloxamer is poloxamer 407.

In other embodiments, the micelle may comprise one or more of PDMS (poly(dimethylsiloxane)), PMA (poly(methacrylic acid)), PAA (poly(acrylic acid)), PNIPAAm (poly(N-isopropylacrylamide)), PAM (polyarylamide), HA (hyaluronic acid), AL (alginate), PVA (poly(vinylalchol)), PDADMAC (poly(diallyldimethylammonium chloride)), NaA (sodium alginate), AAm (acrylamide), NIPAAm (N-isopropylacrylamide), PVME (poly(vinyl methyl ether)), PEG (poly(ethylene glycol)), PPG (poly(propylene glycol), MC (methylcellulose), PDEAEM (poly(N,N-ethylaminoethyl methacrylate), glucose, chitosan, and gelatin.

In some embodiments, the micelles are pentablock copolymers comprising poloxamer 407 and poly((diethylamino)-ethyl methacrylate) (PDEAEM).

In some embodiments, the particles are present in a composition in an amount of about 1 microgram to about 1000 micrograms, about 50 micrograms to about 950 micrograms, about 100 micrograms to about 900 micrograms, about 150 micrograms to about 850 micrograms, about 200 micrograms to about 800 micrograms, about 250 micrograms to about 750 micrograms, about 300 micrograms to about 700 micrograms, about 350 micrograms to about 650 micrograms, about 400 micrograms to about 600 micrograms, or about 450 micrograms to about 550 micrograms. In other embodiments, the particles are present in the immunogenic composition in an amount of about 200 micrograms to about 5000 micrograms. In still further embodiments, the particles are present in the immunogenic composition in an amount of about 1 microgram to about 50 micrograms.

In some embodiments, the one or more immunogenic proteins of a sarbecovirus virus are present in the particles and/or the micelles in an amount about 0.1 micrograms to about 1000 micrograms, about 0.1 micrograms to about 900 micrograms, about 0.1 micrograms to about 800 micrograms, about 0.1 micrograms to about 700 micrograms, about 0.1 micrograms to about 600 micrograms, about 0.1 micrograms to about 500 micrograms, about 0.1 micrograms to about 400 micrograms, about 0.1 micrograms to about 300 micrograms, about 0.1 micrograms to about 200 micrograms, about 0.1 micrograms to about 100 micrograms, or about 0.1 micrograms to about 50 micrograms. In some embodiments, the one or more immunogenic proteins of a sarbecovirus virus are present in the particles in an amount of about 1 microgram to about 15 micrograms, for example, about 1 microgram, about 2 micrograms, about 3 micrograms, about 4 micrograms, about 5 micrograms, about 6 micrograms, about 7 micrograms, about 8 micrograms, about 9 micrograms, about 10 micrograms, about 11 micrograms, about 12 micrograms, about 13 micrograms, about 14 micrograms, or about 15 micrograms. In some embodiments, the one or more immunogenic proteins of a sarbecovirus virus are present in the particles in an amount of about 3 micrograms to about 13 micrograms, or about 5 micrograms to about 10 micrograms. In other embodiments, the one or more immunogenic proteins of the sarbecovirus are present in the particles in an amount of about 2 micrograms to about 50 micrograms. Alternatively, amounts of immunogenic proteins in a composition may be expressed in weight percentage. For example, in 500 micrograms of particles, 5 micrograms of immunogenic proteins comprise 1 wt. %, 10 micrograms of immunogenic proteins comprise 2 wt. %, etc. Accordingly, a given immunogenic protein may comprise about 0.01 wt % to about 10 wt %, or about 0.05 wt % to about 10 wt %, or about 0.1 wt % to about 10 wt %. In other embodiments, a given immunogenic protein may comprise about 1 wt % to about 5 wt %. In other embodiments, a given immunogenic protein may comprise about 0.1 wt %, about 0.5 wt. %, about 1 wt. %, about 1.5 wt. %, about 2 wt. %, about 2.5 wt. %, about 3 wt. %, about 3.5 wt. %, about 4 wt. %, about 4.5 wt. %, or about 5 wt. % of the total weight of the particles or the micelles.

In some embodiments, the pentablock copolymer micelles are present in the immunogenic composition in an amount of about 0.5 milligrams to about 500 milligrams, about 0.5 milligrams to about 400 milligrams, about 0.5 milligrams to about 300 milligrams, about 0.5 milligrams to about 200 milligrams, about 0.5 milligrams to about 100 milligrams, about 0.5 milligrams to about 75 milligrams, about 0.5 milligrams to about 50 milligrams, or about 0.5 milligrams to about 25 milligrams. In some embodiments, the pentablock copolymer micelles are present in the immunogenic composition in an amount of about 0.5 milligram to about 20 milligrams, for example, about 0.5 milligrams, about 1 milligram, about 2 milligrams, about 3 milligrams, about 4 milligrams, about 5 milligrams, about 6 milligrams, about 7 milligrams, about 8 milligrams, about 9 milligrams, about 10 milligrams, about 11 milligrams, about 12 milligrams, about 13 milligrams, about 14 milligrams, about 15 milligrams, about 16 milligrams, about 17 milligrams, about 18 milligrams, about 19 milligrams, or about 20 milligrams. In some embodiments, the pentablock copolymer micelles are present in the immunogenic composition in an amount of about 0.5 milligram to about 1 milligram. In other embodiments, the pentablock copolymer micelles are present in an amount of about 2 milligrams to about 6 milligrams. In some embodiments, the amount of pentablock copolymer micelles present in the immunogenic composition is about 8 milligrams.

In some embodiments, one or more adjuvants are present in the particles or micelles in an amount of about 0.1 micrograms to about 1000 micrograms, about 0.1 micrograms to about 900 micrograms, about 0.1 micrograms to about 800 micrograms, about 0.1 micrograms to about 700 micrograms, about 0.1 micrograms to about 600 micrograms, about 0.1 micrograms to about 500 micrograms, about 0.1 micrograms to about 400 micrograms, about 0.1 micrograms to about 300 micrograms, about 0.1 micrograms to about 200 micrograms, about 0.1 micrograms to about 100 micrograms, or about 0.1 micrograms to about 50 micrograms. In some embodiments, the one or more immunogenic proteins of a sarbecovirus virus are present in the particle in an amount of about 1 microgram to about 15 micrograms, for example, about 1 microgram, about 2 micrograms, about 3 micrograms, about 4 micrograms, about 5 micrograms, about 6 micrograms, about 7 micrograms, about 8 micrograms, about 9 micrograms, about 10 micrograms, about 11 micrograms, about 12 micrograms, about 13 micrograms, about 14 micrograms, or about 15 micrograms. In some embodiments, the one or more immunogenic proteins of a sarbecovirus virus are present in the particle in an amount of about 3 micrograms to about 13 micrograms, or about 5 micrograms to about 10 micrograms.

In another embodiment, an immunogenic composition may comprise copolymers of one or more monomers of 1,8-bis(p-carboxyphenoxy)-3,6-dioxaoctane (CPTEG), 1,6-bis(p-carboxyphenoxy) hexane (CPH), sebacic anhydride (SA), a SA homopolymer, a CPTEG:CPH copolymer, a CPTEG:SA copolymer, a CPH:SA copolymer, a CPH homopolymer, or poly(lactic acid-co-glycolic Acid) (PLGA); pentablock copolymer micelles comprising a poloxamer and poly((diethylamino)-ethyl methacrylate) (PDEAEM); a spike protein (S protein) and a nucleocapsid protein (N protein) of SARS-CoV-2 virus; and an adjuvant comprising cyclic di-guanosine monophosphate (c-di-GMP); wherein a first amount of the S protein, a first amount of the N protein, and a first amount of the adjuvant are encapsulated within the biodegradable particles; and a second amount of the S protein, a second amount of the N protein, and a second amount of the adjuvant are entrapped within the pentablock copolymer micelles.

In another embodiment, an immunogenic composition may comprise copolymers comprising one or more monomers of 1,8-bis(p-carboxyphenoxy)-3,6-dioxaoctane (CPTEG), 1,6-bis(p-carboxyphenoxy) hexane (CPH), sebacic anhydride (SA), a SA homopolymer, a CPTEG:CPH copolymer, a CPTEG:SA copolymer, a CPH:SA copolymer, a CPH homopolymer, or poly(lactic acid-co-glycolic Acid) (PLGA); pentablock copolymer micelles comprising poloxamer 407 and poly((diethylamino)-ethyl methacrylate) (PDEAEM); a spike protein (S protein) and a nucleocapsid protein (N protein) of SARS-CoV-2 virus; and an adjuvant comprising cyclic di-guanosine monophosphate (c-di-GMP); wherein a first amount of the S protein and the N protein, and a first amount of the adjuvant is encapsulated within the particles; and a second amount of the S protein and the N protein, and a second amount of the adjuvant is entrapped within the pentablock copolymer micelles.

In another embodiment, an immunogenic composition may comprise biodegradable a polyanhydride particles comprising 1,8-bis(p-carboxyphenoxy)-3,6-dioxaoctane (CPTEG) and 1,6-bis(p-carboxyphenoxy) hexane (CPH) copolymers in a molar ratio of about 20:80; pentablock copolymer micelles comprising a poloxamer and poly((diethylamino)-ethyl methacrylate) (PDEAEM); a spike protein (S protein) and a nucleocapsid protein (N protein) of SARS-CoV-2 virus; and an adjuvant comprising cyclic di-guanosine monophosphate (c-di-GMP); wherein a first amount of the S protein and the N protein, and a first amount of the adjuvant is encapsulated within the particles; and a second amount of the S protein and the N protein, and a second amount of the adjuvant is entrapped within the pentablock copolymer micelles.

In some embodiments, both the first amount of the S protein and the N protein and the second amount of the S protein and the N protein is about 0.1 micrograms to about 500 micrograms; and both the first amount of the adjuvant and the second amount of the adjuvant is about 0 micrograms to about 500 micrograms. In some embodiments, the first amount of the adjuvant encapsulated within the particles is 0.

In some embodiments, the first amount of the S protein and the N protein is about 5 micrograms to about 10 micrograms, the first amount of the adjuvant is about 0 micrograms to about 10 micrograms. In some embodiments, the second amount of the S protein and the N protein is about 5 micrograms to about 10 micrograms, and a second amount of the adjuvant entrapped within the pentablock copolymer micelles is about 10 micrograms to about 20 micrograms.

In some embodiments, the adjuvant further comprises an adjuvant other than 1,8-bis(p-carboxyphenoxy)-3,6-dioxaoctane (CPTEG) and 1,6-bis(p-carboxyphenoxy) hexane (CPH).

The disclosure also provides for methods of inducing an immune response against a sarbecovirus infection in a subject in need thereof comprising administering to the subject an effective amount of an immunogenic composition described herein, thereby inducing an immune response. In some embodiments, the immune response comprises both a local immune response and systemic protective immune response against the sarbecovirus.

In some embodiments, the preferred mode of administration is nasal administration. In some embodiments, particles can persist in the lungs for one or more months, gradually releasing encapsulated contents as the particles degrade. Specifically, the 20:80 CPTEG:CPH particles are visible in the lungs for at least 63 days (Haughney et al., Nanoscale 6 (22), 13770-8 (2014)). The hydrophobic chemistry of this formulation (high CPH content), coupled with the thermal properties of the copolymer enable facile synthesis of particles that have highly effective persistence time in the lungs. The persistence time is highly advantageous because reduced CPH content in the copolymer may result in shorter persistence (lessening the depot effect) and increased CPH content may lead to tolerance and/or difficulty in preparing nanoparticle-based formulations suitable for intranasal delivery. The 20:80 CPTEG:CPH formulation therefore provides a balance of advantageous persistence kinetics as well as facile synthesis suitable for intranasal delivery. In other embodiments, administration may be via parenteral and/or nasal administration.

The immunogen-loaded particles may be prepared by the solid/oil/oil solvent displacement technique similar to the method reported by Ulery et al. (2011) PLoS ONE 6, E17642). The polymer is dissolved in an organic solvent such as methylene chloride (typically about 20-30 mg/mL). Any suitable solvent can be used to dissolve the polymer. Suitable examples include chlorinated organic solvents such as methylene chloride, chloroform, and carbon tetrachloride. The immunogen is lyophilized into a dry powder. The immunogen is added to the polymer solution in a glass vial and immediately dispersed by agitation with a handheld homogenizer or sonicator.

The polymer solution may be precipitated by transferring to a container of cold pentane (non-solvent). The mixture may then be rapidly stirred with an impeller to extract the solvent. The particles may then be isolated by filtration. The particles may then be rinsed with additional non-solvent and dried under vacuum to afford a free-flowing powder.

Particles also may be prepared by modifications of the solvent removal technique described above. Suitable modifications may include, but are not limited to a water/oil/oil emulsion, a water/oil/water/emulsion, or spray drying techniques well known to those skilled in the art.

The pentablock copolymer micelles may be prepared by atom transfer radical polymerization as previously described (Adams et al., Macromol Chem Phys 214, 1321-1325 (2013); Determan et al., Polymer 46, 6933-6946 (2005)). Briefly, prepared macroinitiator and DEAEM monomer are used to synthesize the pentablock copolymer by utilizing copper (I) oxide nanoparticle catalysts and N-propylpyrilidine methanamine as complexing ligands.

The particles, immunogens, and adjuvants described herein may be used to prepare pharmaceutical compositions, for example, by combining the particles and adjuvants with a pharmaceutically acceptable diluent, excipient, or carrier. The particles may be added to a carrier in the form of a salt or solvate. Examples of pharmaceutically acceptable salts are organic acid addition salts formed with acids that form a physiological acceptable anion, for example, tosylate, methanesulfonate, acetate, citrate, malonate, tartrate, succinate, benzoate, ascorbate, α-ketoglutarate, and γ-glycerophosphate. Suitable inorganic salts also may be formed, including hydrochloride, halide, sulfate, nitrate, bicarbonate, and carbonate salts, sodium chloride, calcium chloride, sodium phosphate, monosodium glutamate, and aluminum salts (e.g., aluminum hydroxide, aluminum phosphate, alum (potassium aluminum sulfate), or a mixture of such aluminum salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

To control tonicity, it is preferred to include a physiological salt, such as a sodium salt. Sodium chloride (NaCl) is preferred, which may be present at between 1 and 20 mg/mL, e.g., about 10±2 mg/mL NaCl. Other salts that may be present include potassium chloride, potassium dihydrogen phosphate, disodium phosphate dehydrate, magnesium chloride, calcium chloride, etc.

Immunogenic compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g., 6.5 and 7.5, or between 7.0 and 7.8. The composition is preferably sterile. The composition is preferably non-pyrogenic e.g., containing <1 EU (endotoxin unit, a standard measure) per dose, and preferably <0.1 EU per dose. The composition is preferably gluten free.

In certain embodiments, the immunogenic composition includes as an excipient such as water or phosphate buffered saline.

Immunogenic compositions (such as a vaccine) may be formulated and administered in accordance with standard techniques well known to those skilled in the art. For example, a vaccine can be prepared by any suitable method, such as the methods described by Franchini et al., (International Patent Application No. PCT/US2003/035499), Kipper et al., (U.S. Pat. No. 7,858,093), or Caputa et al. (U.S. Pat. No. 5,554,371, for example, at col. 7). Other useful techniques can be used such as those described by Cleary (U.S. Pat. No. 5,846,547, for example, at col. 6-7). One preferred immunogenic composition may comprise one or more copolymers forming a biodegradable particle, the copolymers including 1,8-bis(p-carboxyphenoxy)-3,6-dioxaoctane (CPTEG) and 1,6-bis(p-carboxyphenoxy) hexane (CPH) in a molar ratio of about 20:80, one or more immunogenic proteins of a sarbecovirus, and optionally, one or more adjuvants encapsulated within an interior of the particle. In other embodiments, immunogenic compositions may further comprise pentablock copolymer micelles, and a second amount of the one or more immunogenic proteins of sarbecovirus and a second amount of the one or more adjuvants entrapped within the pentablock copolymer micelles.

The immunogenic compositions may include particles that comprise a mixture of various immunogens, such as those listed above. Alternatively, immunogenic compositions may include a mixture of particles, where each of the particles include a single immunogen. For example, with respect to the immunogen, a first population of particles include only the S protein of SARS-CoV-2 and a second population of particles include only the N protein of SARS-CoV-2.

Immunogenic compositions or particles may be administered to a subject by a variety of routes. These include, but are not limited to, intranasal, intratracheal, oral, intradermal, intramuscular, intraperitoneal, transdermal, intravenous, conjunctival, and subcutaneous routes. In some embodiments, a composition is formulated for topical administration, for example, for application to the skin. In a preferred embodiment, the route of administration is nasal, e.g., as part of a nasal spray or dry powder. In some embodiments, the administering route comprises intranasal administration and/or parenteral administration. Parenteral administration includes one or more of subcutaneous, intramuscular, intravenous, and intrathecal administration.

It is further contemplated that an immunogenic composition of the invention may be administered through a combination of administration routes, or one or more administrations of composition using the same route of delivery. For example, an intranasal dosing may be delivered to the subject prior to a parenteral administration (e.g., intramuscular dosing or subcutaneous administration). Alternatively, an intranasal dosing may be delivered to the subject subsequent to intramuscular dosing or subcutaneous (parenteral) administration.

In another example, the particles as described herein may be administered intranasally. In another example, both the particles and pentablock copolymer micelles are administered parenterally. In still another example, the particles may be administered intranasally and the pentablock copolymer micelles, with or without particles, are delivered parenterally.

For administration to a subject, a first immunogenic composition may be administered to the individual (prime) and then after a period of time, a second immunogenic composition (the same or different than the first immunogenic composition) may be administered to the individual (boost). Administration of the boosting composition is generally weeks or months after administration of the priming composition, preferably about 2-3 weeks or 4 weeks, or 8 weeks, or 16 weeks, or 20 weeks, or 24 weeks, or 28 weeks, or 32 weeks. In one embodiment, the boosting composition is formulated for administration about 1 week, or 2 weeks, or 3 weeks, or 4 weeks, or 5 weeks, or 6 weeks, or 7 weeks, or 8 weeks, or 9 weeks, or 16 weeks, or 20 weeks, or 24 weeks, or 28 weeks, or 32 weeks after administration of the priming composition. Priming and boosting may be accomplished through one or more of the means disclosed herein (e.g., a nasal prime and nasal boost etc.). The route of administration of the prime and boost need not be the same (e.g., a nasal prime and intramuscular boost etc.).

The first and second immunogenic compositions may be, but need not be, the same composition. Thus, in one embodiment of the present invention, the step of administering the immunogenic composition to a subject comprises administering a first immunogenic composition, and then at a later time, administering a second immunogenic composition. For example, the first immunogenic composition (prime) may comprise biodegradable particles comprise one or more polymers, wherein the polymers comprise one or more monomers of 1,8-bis(p-carboxyphenoxy)-3,6-dioxaoctane (CPTEG), 1,6-bis(p-carboxyphenoxy) hexane (CPH), sebacic anhydride (SA), SA homopolymer, a CPTEG:CPH copolymer, a CPTEG:SA copolymer, a CPH:SA copolymer, a CPH homopolymer, or a polyester copolymer; at least one immunogenic protein of a sarbecovirus; and at least one adjuvant; wherein a first amount of the immunogenic protein and a first amount of the adjuvant are encapsulated within the biodegradable particles. The second immunogenic composition (i.e., the booster) may comprise biodegradable particles comprising one or more polymers, wherein the polymers comprise one or more monomers of 1,8-bis(p-carboxyphenoxy)-3,6-dioxaoctane (CPTEG), 1,6-bis(p-carboxyphenoxy) hexane (CPH), sebacic anhydride (SA), a SA homopolymer, a CPTEG:CPH copolymer, a CPTEG:SA copolymer, a CPH:SA copolymer, a CPH homopolymer, or a polyester copolymer; at least one immunogenic protein of a sarbecovirus; and at least one adjuvant; and pentablock copolymer micelles; wherein a first amount of the immunogenic protein and a first amount of the adjuvant are encapsulated within the biodegradable particles, and wherein a second amount of the immunogenic protein, a second amount of the adjuvant, or both are entrapped within one or more of the pentablock copolymer micelles. It is also contemplated that the first immunogenic composition may be used as the boosting administration and the second composition may be used as the priming administration. Alternatively, a third immunogenic composition, for example, comprising pentablock copolymer micelles (no particles), and a second amount of the one or more immunogenic proteins of sarbecovirus and a second amount of the one or more adjuvants entrapped within the pentablock copolymer micelles may be used as either the prime or boost instead of one of the first or second immunogenic compositions described above. In some embodiments, the same immunogenic composition may be used as both the prime and boost.

The separate compositions may be administered via the same or separate routes of administration. For example, the first composition may be administered intranasally or parenterally while the second composition is administered parenterally. Generally, an immunogenic composition comprising the particles is administered intranasally and/or parenterally while an immunogenic composition comprising the particles and the pentablock copolymer micelles (or just the pentablock copolymer micelles) are administered parenterally.

It is contemplated that any embodiment discussed in this specification may be implemented with respect to any method, kit, reagent, or composition, and vice versa. Furthermore, the compositions may be used to achieve the methods of invention.

Embodiments of the immunogenic composition of the invention also may include material for a single administration or may include material for multiple administrations (i.e., a ‘multidose’ kit). The inclusion of a preservative is preferred in multidose arrangements. As an alternative (or in addition) to including a preservative in multidose compositions, the compositions may be stored in a container having an aseptic adaptor for removal of material.

The following examples are intended to illustrate the above invention and should not be construed as to narrow its scope. One skilled in the art will readily recognize that the examples suggest many other ways in which the invention could be practiced. It should be understood that numerous variations and modifications may be made while remaining within the scope of the invention.

### EXAMPLES

### Example 1. Next-Generation Nanovaccines Induce Durable Antibody Responses, Generate Antigen-Specific T Cells, and Protect Against SARS-CoV-2 Lethal Challenge in Mice

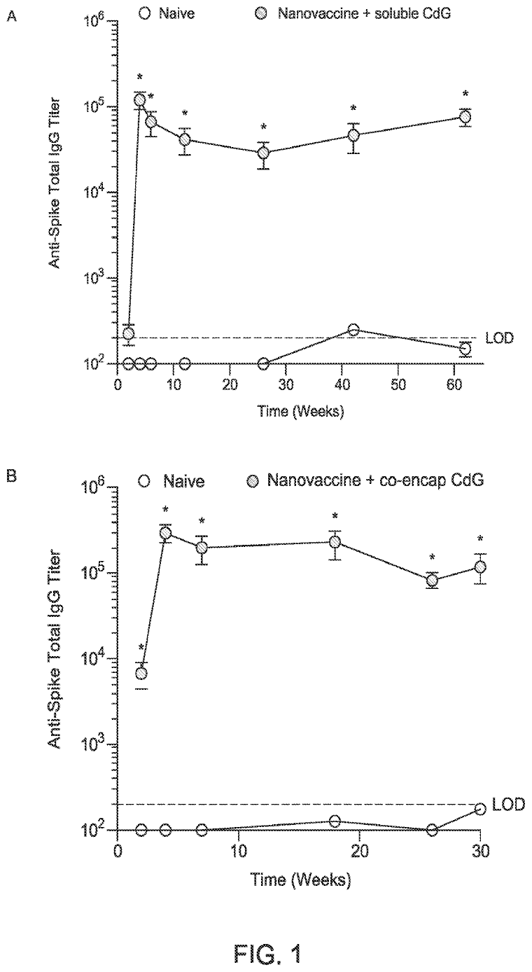

SARS-CoV-2 nanovaccine induced high titer, long-lived antibody responses against spike. A SARS-CoV-2 nanovaccine formulation containing S and N antigens co-encapsulated into particles or polyanhydride nanoparticles delivered with PBC micelles, soluble S and N antigen, and soluble CdG was designed (Table 2). To assess the immunogenicity of this combination nanovaccine, mice received two subcutaneous immunizations administered three weeks apart; systemic anti-S IgG antibody titers were measured over 62 weeks post-vaccination. While no significant anti-S IgG antibody titer was detected two weeks after the primary immunization, the administration of a booster on day 21 resulted in a significant (p<0.03) increase in titer by week 4 (FIG. 1A). Nanovaccine-immunized animals maintained anti-S IgG antibody titers ≥10⁴ for at least 62 weeks.

There is evidence that encapsulation of CdG within particles enhances systemic antibody responses to vaccine antigens (Hanson et al., J Clin Invest 125 (6), 2532-2546 (2015)), and that co-encapsulation of antigen and adjuvant within the same carrier can enhance antigen internalization and cognate antigen presentation to T cells, thereby facilitating adaptive immune responses (Blander and Medzhitov, Nature 440 (7085), 808-812 (2006)). Based on this, we hypothesized that the adaptive immune response may be enhanced by co-encapsulating the antigens (S and N) and co-adjuvant (CdG) within the polyanhydride nanoparticles. Therefore, mice were vaccinated with a SARS-CoV-2 nanovaccine in which 50% of the CdG co-adjuvant was encapsulated into the polyanhydride nanoparticles with S and N antigens (Table 2). In support of our hypothesis, animals vaccinated with co-encapsulated CdG did indeed develop anti-S antibody titers of approximately 10⁴ as early as two weeks after the first immunization (FIG. 1B), in contrast to mice vaccinated with the previous nanovaccine formulation, which contained the CdG co-adjuvant as a soluble bolus (FIG. 1A). In addition, following a booster at week three, animals immunized with co-encapsulated CdG and antigens developed similar long-lived, high titer antibody responses (~10⁴-10⁶) through at least 30 weeks, suggesting that although both formulations induced similar long-lived antibody responses, co-encapsulation of the co-adjuvant may be important for rapid development of humoral immunity.

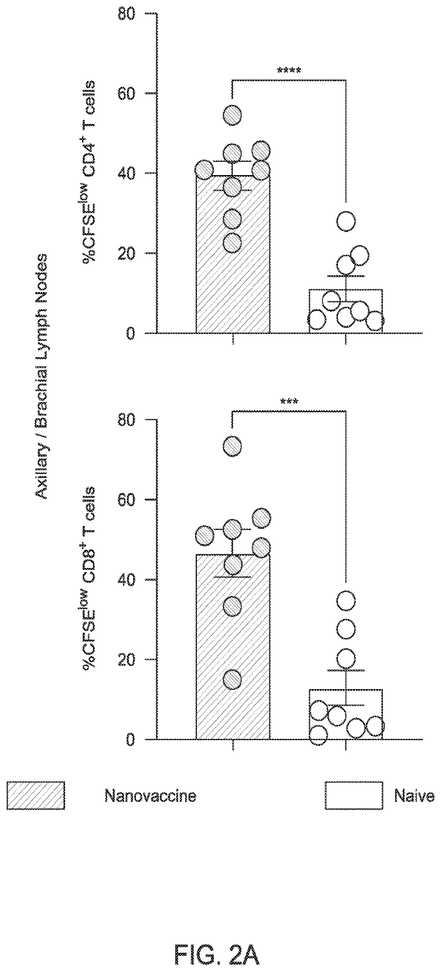

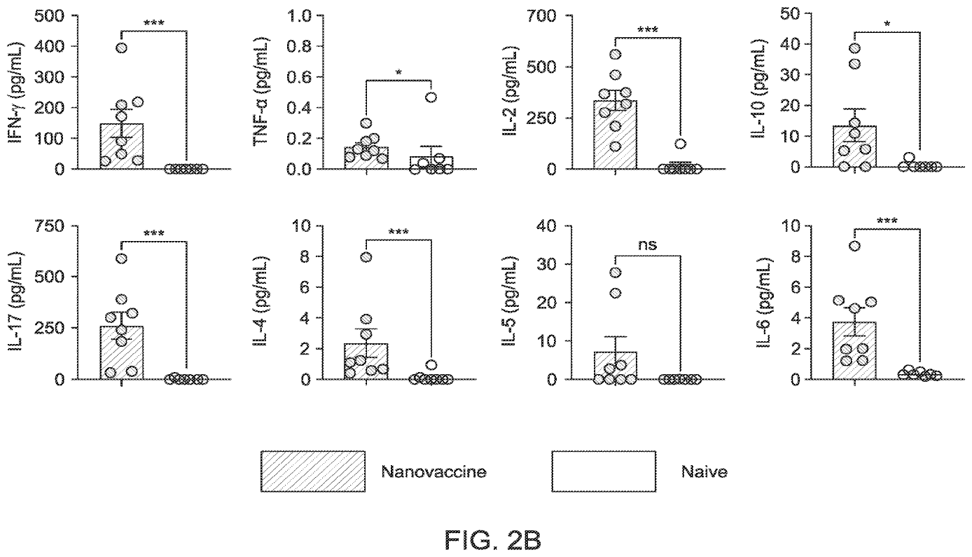

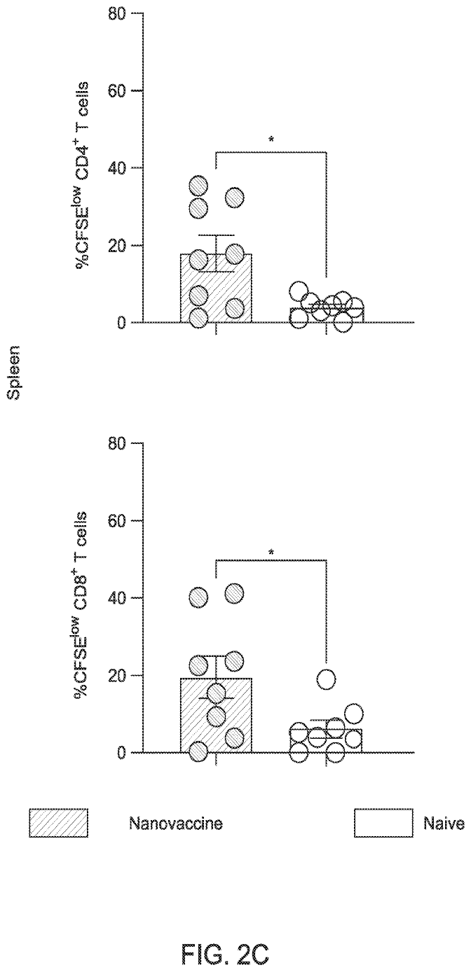

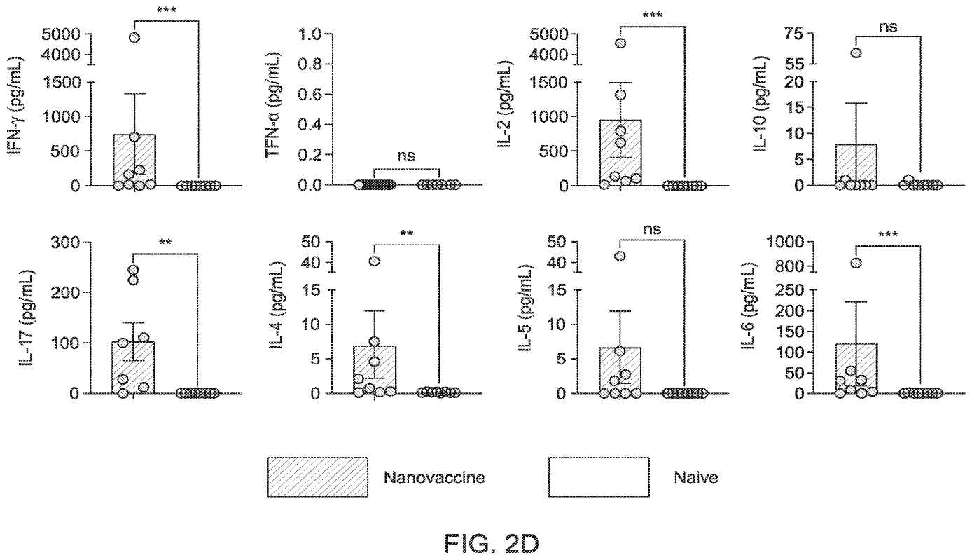

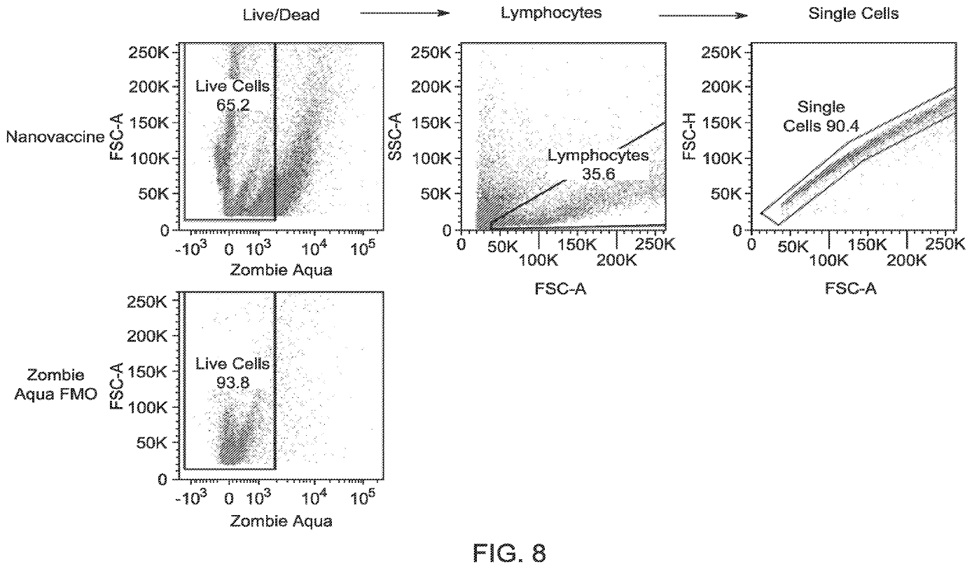

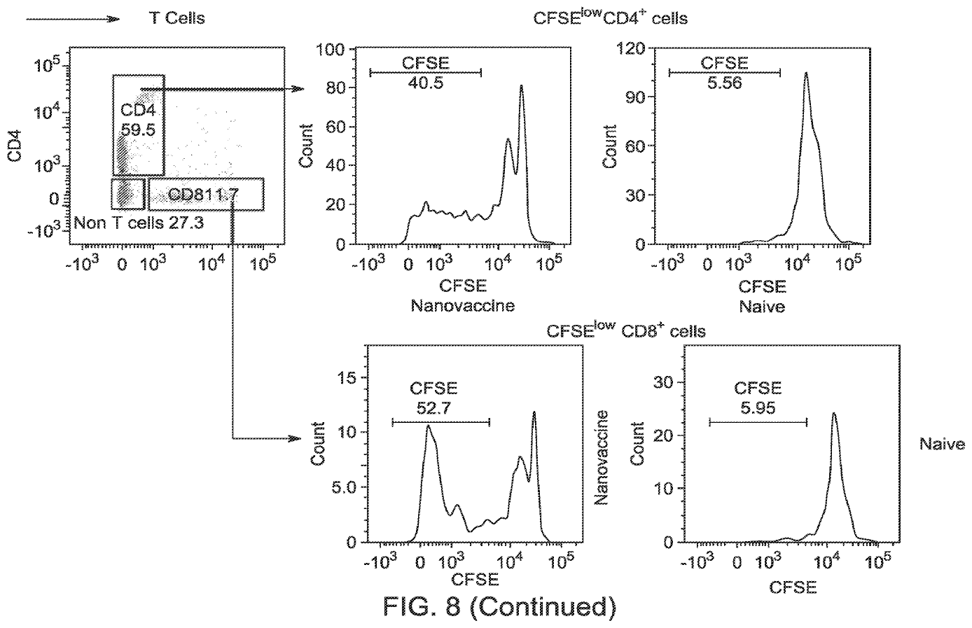

Mice immunized with SARS-CoV-2 nanovaccine developed antigen-specific CD4⁺ and CD8⁺ T cell responses. In addition to durable antibody responses, it has been suggested that cell-mediated immunity (i.e., T cells) also contributes to reducing disease severity and providing protection against SARS-CoV-2 infections. To examine systemic T cell responses induced by SARS-CoV-2 nanovaccines in mice, axillary/brachial lymph nodes and spleens were harvested from mice 45 days post-immunization with the nanovaccine co-encapsulating CdG. Cells were labeled with carboxyfluorescein succinimidyl ester (CFSE) and stimulated ex vivo for five days with S and N antigens; subsequently, flow cytometry was used to measure antigen-specific T cell proliferation (see FIG. 8 for flow gating strategy). Lymphocytes from mice immunized with the nanovaccine demonstrated greater proliferation (i.e., CFSE^{low}) of both CD4⁺ and CD8⁺ T cells as compared to lymphocytes from naïve mice, indicating the presence of antigen-specific T cells in both the draining lymph nodes and spleens (FIGS. 2A & C). Additionally, cell culture supernatants harvested after five days of restimulation with S and N antigen were analyzed for the presence of secreted cytokines, in particular, Th₁ cytokines IFN-γ and TNF-α, Th₂ cytokines IL-4 and IL-5, and Th₁₇ cytokine IL-17, which would indicate cytokines secreted by T cells as part of the recall response to antigen restimulation. We also included multiple other cytokines as part of our standard panel (FIGS. 2B & D). Following in vitro restimulation, lymphocytes from the draining lymph nodes of combination nanovaccine-immunized animals were found to secrete IL-2, IFN-γ, and IL-17, indicating induction of cell-mediated immunity (FIG. 2). Splenocytes from mice administered the nanovaccine demonstrated similar profiles of cytokine secretion as the draining lymph nodes. Altogether, these results indicate that the SARS-CoV-2 nanovaccine induced antigen-specific CD4⁺ and CD8⁺ T cells that could be detected 45 days post-immunization in mice.

Mice immunized with SARS-CoV-2 nanovaccine developed virus neutralizing antibody responses. Noting that mice immunized with the SARS-CoV-2 nanovaccine generated rapid and prolonged serum IgG responses to S antigen (as shown in FIG. 1B) as well as antigen-specific T cell responses (FIG. 2), the antigen dose of the nanovaccine formulation was further optimized in order to enhance the overall serum IgG response to S, as well as serum viral neutralization capabilities. To this end, we assessed systemic anti-S IgG antibody responses post-vaccination in a SARS-CoV-2 susceptible mouse model (K18-hACE2 mice) by comparing serum responses in mice immunized with a nanovaccine formulation containing a 1× antigen dose (i.e., 10 μg of each S and N as presented in FIGS. 1B and 2) to mice immunized with a nanovaccine formulation containing a 2× antigen dose (i.e., 20 μg of each S and N); this 2× antigen dose nanovaccine contained an equivalent amount of polyanhydride nanoparticles, PBC micelles, and CdG compared to the 1× antigen dose nanovaccine. Briefly, K18-hACE2 mice received two immunizations three weeks apart of either the 1× or 2× antigen dose nanovaccine. Five weeks after the primary immunization, serum was collected and analyzed via ELISA against the S protein. For viral neutralization, a surrogate virus neutralization test (sVNT, wild-type RBD, GenScript Biotech Corp.) was used to determine the ability of serum antibody to inhibit binding of S RBD with the human ACE2 receptor.

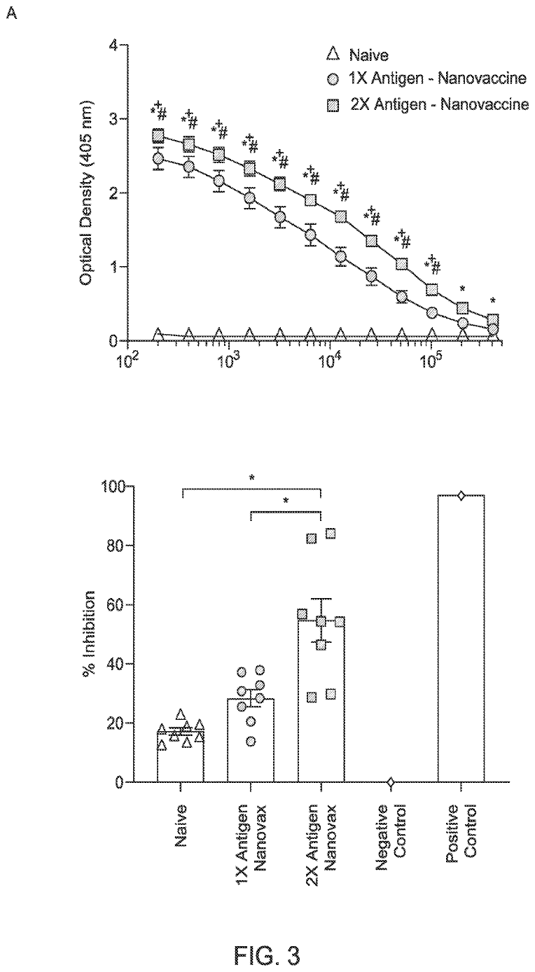

Mice immunized with both nanovaccine formulations demonstrated high titer, serum antibody responses to the wild-type, full-length S antigen, however the 2× antigen dose nanovaccine induced significantly increased serum IgG antibody responses 35 days post-immunization compared to the 1× antigen dose nanovaccine (FIG. 3A). In agreement with serum IgG responses to full-length S antigen, serum from mice immunized with the 2× antigen dose nanovaccine achieved ~55% inhibition of S RBD binding to ACE2, which was significantly increased compared to serum from 1× antigen dose nanovaccine and naïve groups (FIG. 3B). Interestingly, despite the moderately high serum IgG response to wild-type, full-length S protein, serum from mice receiving 1× antigen dose nanovaccine did not significantly inhibit S RBD binding to ACE2 compared to serum from naïve mice.

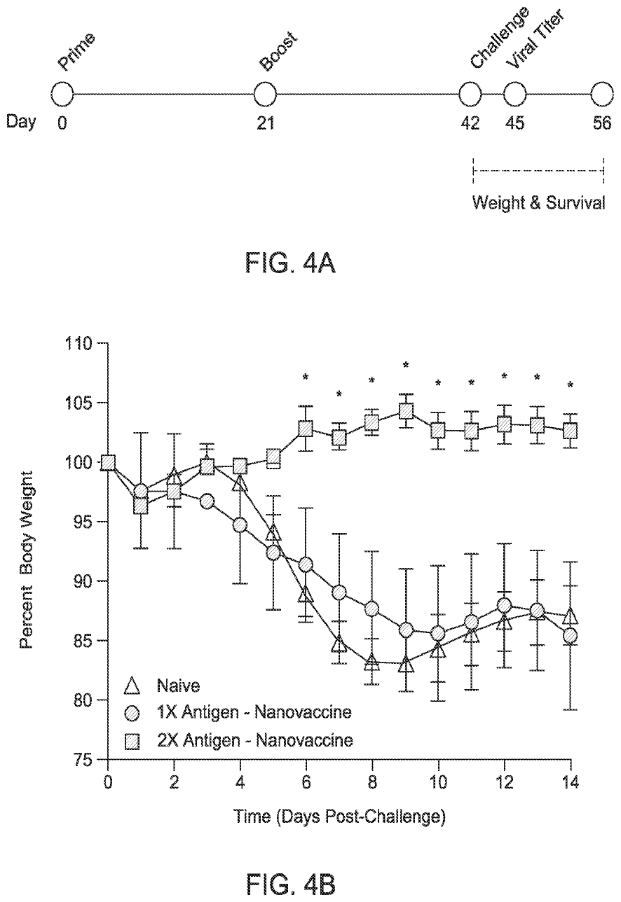

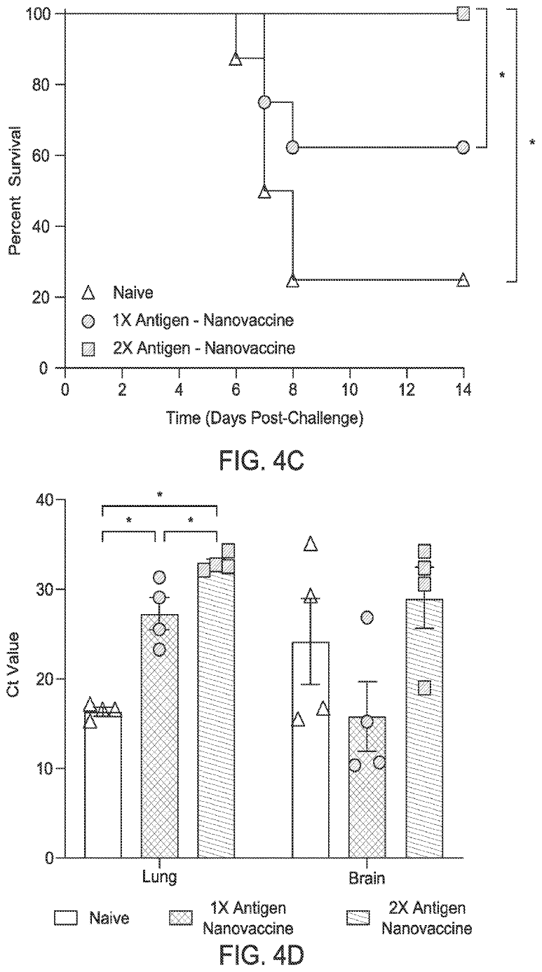

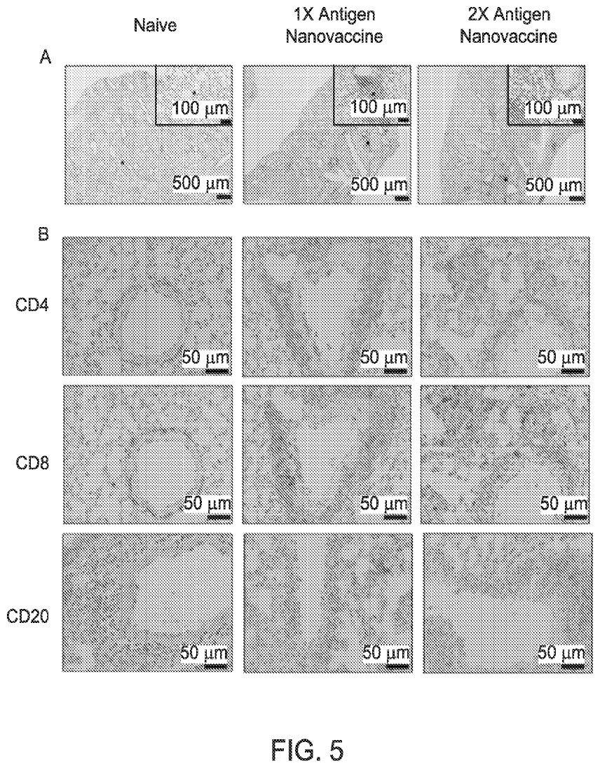

Optimized SARS-CoV-2 nanovaccine protected mice against lethal SARS-CoV-2 viral challenge. Based on the improved serum IgG responses observed from the 2× antigen dose nanovaccine (i.e., anti-S IgG titers and S RBD neutralization responses) as well as the ability of the nanovaccines to induce antigen-specific T cell responses, we sought to determine the ability of this optimized nanovaccine to protect against lethal challenge of SARS-CoV-2 virus. K18-hACE2 mice receiving a prime-boost regimen of the 1× or 2× antigen dose nanovaccine were challenged with 2.5×10⁴ PFU of SARS-CoV-2 (FIG. 4A), representing a challenge dose that induces lesions resembling that of human disease pathology. Throughout the challenge study, mice receiving the 2× antigen dose nanovaccine maintained or slightly gained weight after infection, in contrast to naïve mice or those vaccinated with the 1× antigen dose nanovaccine, which lost ~15% of their body weight post-challenge (FIG. 4B). Additionally, the 2× antigen dose nanovaccine provided 100% protection following lethal challenge compared to 60% survival of animals vaccinated with the 1× antigen dose nanovaccine, while 80% of naïve animals succumbed to lethal challenge (FIG. 4C). All animals presented with similar viral loads within the brain tissue five days post-challenge; however, both nanovaccines significantly reduced viral load (as indicated by increased Ct value) within the lungs compared to naïve mice (FIG. 4D). Additionally, lung tissue harvested five days post-challenge was fixed in formalin, section, and stained with H&E before evaluation by a board-certified veterinary pathologist. While all lung tissue sections depicted mild interstitial pneumonia, those of immunized mice presented with more pervasive perivascular inflammatory cells composed of lymphocytes, plasma cells, and macrophages suggesting recruitment of immune cells (FIG. 5A, FIG. 9). Additionally, immunohistochemical (IHC) analysis of separate lung tissue sections was performed by staining with anti-CD4, anti-CD8α, and anti-CD20 to detect the presence of T and B lymphocytes. The perivascular and interstitial lymphocytic population in the lung sections indicated approximately equal numbers of lymphocytes with membranous CD4 and CD8 expression, and approximately 5-10% CD20-positive lymphocytes (FIG. 5B). The IHC analysis demonstrated that there was an expansion of B and T cells in the perivascular spaces (i.e., bronchus-associated lymphoid tissue or BALT) in the lungs of the vaccinated mice. While the IHC also detected the presence of B and T cells in the lungs of the naïve, infected mice, these cells expanded the vascular tunica adventitia, occasional tunica media, and alveolar septa. The IHC analysis also suggested that there was a more prominent T cell response in the lungs than a B cell response in the lungs of the vaccinated mice following infection with SARS-CoV-2.

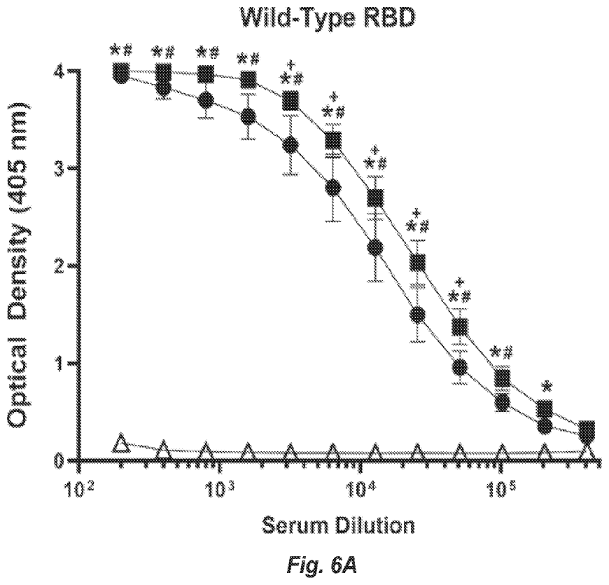

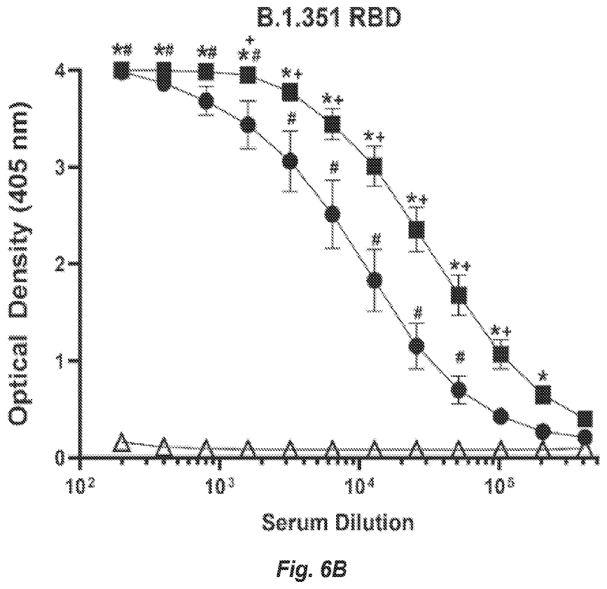

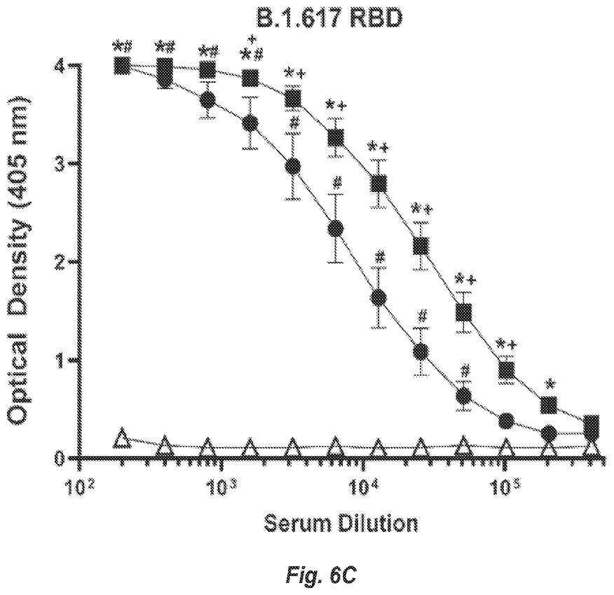

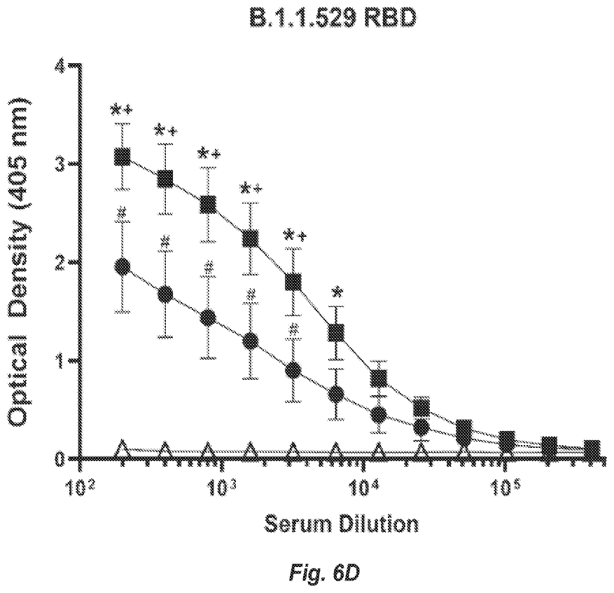

SARS-CoV-2 nanovaccine induced cross-reactive antibody to multiple viral variants. Given the emergence of SARS-CoV-2 variants with greater transmissibility, we assessed cross-reactivity of serum from K18-hACE2 mice collected 35 days after primary immunization (seven days prior to lethal challenge as shown in FIG. 6) to the S protein RBD antigens from multiple SARS-CoV-2 variants, including Omicron variant B.1.1.529, which has 15 mutations in spike RBD compared to wild-type Wuhan-Hu-1.

Mice immunized with both nanovaccine formulations demonstrated high titer, systemic antibody responses to the wild-type S RBD (FIG. 6), as well as cross-reactive antibodies to S RBDs derived from Beta (B.1.351), B.1.617 (parent lineage of Delta and Kappa), and Omicron variant (B.1.1.529) variants through at least 35 days after primary immunization (FIG. 6B-D). It was noted that the 2× antigen dose nanovaccine formulation consistently resulted in higher antibody responses to each of the RBDs assessed compared to the responses induced by the 1× antigen nanovaccine.

The emergence of SARS-CoV-2 highlighted the need for the development and deployment of vaccines and vaccine platforms that improve pandemic preparedness. While currently approved vaccines against SARS-CoV-2 have aided in reducing viral transmission as well as disease severity, there is still room for improvement. First, the monovalent Pfizer (BNT162b2) and Moderna (mRNA-1273) mRNA vaccines originally deployed have been shown to induce antibody responses that wane after approximately 6 months post-boost, with a concomitant significant decrease in neutralizing antibody. Multiple boosters and even annual immunizations may also be required to maintain immunity, likely reducing compliance. Additionally, current vaccines have been designed to only target the S antigen, which has shown to provide subpar immunity against emergent viral variants if selective pressure (i.e., host immunity) causes the viral S protein to mutate away from preexisting, circulating antibodies, as was the case with both the Beta (B.1.351) and Omicron (B.1.1.529) variants (Jackson et al., Nat. Rev. Mol. Cell. Biol. 23 (1) (2022) 3-20; Fan et al., Signal Transduct. Target. Ther. 7 (1) (2022) 141.). Therefore, there is a need for vaccines that can overcome these limitations and provide robust, broad immunity against multiple variants for extended periods of time.

With this in mind, we sought to capitalize on the inherent adjuvant properties of our versatile, combination nanovaccine platform composed of polyanhydride nanoparticles, PBC micelles, and co-adjuvants (CdG, in this case). These particles, which have been shown to persist at the injection site for up two weeks, activate both the cellular and humoral arms of the immune system to provide rapid and durable antibody and T cell immunity to multiple strains of influenza A virus. Similarly, the nanovaccine formulation against SARS-CoV-2 included two antigens, S and N, in order to provide both B cell epitopes to aid in generating antibody responses, as well as more conserved T cell epitopes to induce broad, cellular immunity to emerging SARS-CoV-2 variants. The results of this work demonstrated that prime-boost vaccination with SARS-CoV-2 nanovaccines 1) induced serum anti-S IgG responses capable of binding broadly to variant S RBD domains (FIG. 6), displayed S RBD neutralizing capability (FIG. 3), and maintained long-lived anti-S IgG levels (FIG. 1B), 2) induced antigen-specific CD4⁺ and CD8⁺ T cell responses (FIG. 2), and 3) ultimately provided complete protection against a lethal challenge of SARS-CoV-2 virus (FIG. 4).

Initial formulations of the combination nanovaccine contained polyanhydride nanoparticles encapsulating S and N antigens, PBC micelles associated with S and N antigens, and a soluble bolus of the CdG co-adjuvant, which induced circulating anti-S IgG responses (titer ~5×10⁴) that lasted for at least 62 weeks post-immunization (FIG. 1). As serum anti-S IgG responses were not detected after the primary vaccination, an optimized formulation was designed where half of the CdG co-adjuvant was co-encapsulated into the polyanhydride nanoparticles along with antigen. The rationale was based on evidence that 1) it was previously shown that encapsulation of CdG into lipid nanoparticles enhanced humoral and T cell responses and 2) co-encapsulation of antigen and adjuvant within the same carrier can enhance antigen internalization and cognate antigen presentation to T cells, thereby enhancing adaptive immune responses. Consistent with this approach, vaccination with the nanovaccine co-encapsulating S, N, and CdG induced a more rapid anti-S serum IgG response that was detectable as early as 14 days (FIG. 1B) and maintained this response for at least 30 weeks indicating the importance of co-encapsulating antigen and adjuvant for boosting and maintaining systemic humoral immunity against antigens.

When comparing the performance of the 1× antigen dose and 2× antigen dose nanovaccines (which both contain co-encapsulated CdG), the 2× antigen dose nanovaccine increased the serum anti-S IgG antibody response to wild-type full-length S antigen, as well as significantly higher serum anti-S RBD neutralizing antibody (FIG. 3), and generated the highest cross-reactive antibody responses to S RBD from multiple SARS-CoV-2 variants (FIG. 6). Thus, it correlates that the increased serum anti-S and anti-RBD IgG antibody responses induced with the 2× antigen dose nanovaccine were associated with greater neutralizing antibody responses compared to the 1× antigen dose nanovaccine.

Although a direct correlate of protection has yet to be identified for humans, there is growing evidence that anti-S antibody levels (overall and neutralizing) correlate with reduced disease severity. The diminished binding to RBD from the Omicron (B.1.1.529) variant compared to wild-type RBD (FIG. 6) is consistent with reports of decreased antibody binding due to the high degree of mutations present in the S RBD compared to the wild-type strain; these mutations affect the variant's ability to evade antibodies in vaccinated individuals and facilitate breakthrough infections, highlighting the potential importance developing vaccines that include multiple antigens with conserved epitopes to aid in maintaining immunity against viral variants.

In addition to humoral immunity correlates of protection against SARS-CoV-2, there is evidence that suggests that T cells play a critical role in reducing severity of disease and aiding in immune protection from SARS-CoV-2. Previous work has shown CD8⁺ T cells with specificity to conserved SARS-CoV-2 epitopes correlate with mild disease, suggesting the importance of T cells in protection. In addition, T cell responses in vaccinated individuals appear to be durable (i.e., ≥8 months) and conserved against S antigen across viral variants including Omicron. Furthermore, during the 2003 SARS-CoV outbreak, memory T cells were detectable for 11 years following the outbreak while antibody levels were undetectable after two years, suggesting that T cells may be important for aiding long term protective immunity against SARS-CoV-2. We have previously reported that nanovaccines containing polyanhydride nanoparticles are capable of inducing robust cell-mediated immunity, characterized by CD4⁺ and CD8⁺ T cell responses that improved protective immune responses against heterologous influenza A virus infection, as well as anti-tumor CD8⁺ T cell responses that limited tumor progression. Consistent with those studies, in this work, mice vaccinated with the SARS-CoV-2 nanovaccine demonstrated antigen-specific CD4⁺ and CD8⁺ T cells that proliferated following restimulation, as well as elevated levels of cytokines that play a role in driving cell-mediated immunity (FIG. 2). In this regard, the histopathological evaluation of the lungs (FIG. 5, FIG. 9) indicated that there was an increase in perivascular cuffing in the lungs of the vaccinated mice suggesting the increased presence of lymphocytes. Further studies using this platform will more closely evaluate the T cell response to SARS-CoV-2 nanovaccines, and compare additional routes of immunization, such as intranasal.

As further evidence for evaluating the role of intranasal immunization, Afkhami et al. showed that intranasal administration using an adenoviral-vectored vaccine comprising spike S1 domain, nucleocapsid, and RdRp antigens generated greater anti-S neutralizing antibody responses compared to intramuscular vaccination, induced lung CD8⁺ tissue-resident memory T cells, and provided a high level of protection in K18-hACE2 mice following lethal challenge with SARS-CoV-2. Previous work from our lab has shown that intranasal immunization with a polyanhydride nanovaccine against influenza A virus can induce lung tissue-resident memory CD4⁺ and CD8⁺ T cells, local, systemic antibody responses against influenza A virus, and protected against homologous and heterologous influenza challenge (Zacharias et al., Front Immunol 9 (2018) 1953), therefore further investigation into intranasal immunization with polyanhydride nanovaccines is warranted.

The optimized 2× antigen dose nanovaccine formulation completely protected animals against a lethal challenge dose of SARS-CoV-2 virus previously reported to more closely recapitulate lung disease observed in humans, indicating that optimizing the nanovaccine formulation to increase serum anti-S IgG response and anti-S neutralizing antibody responses correlated with protection (FIG. 4). Interestingly, although mice receiving the 1× antigen dose nanovaccine did not produce significant neutralizing antibody titers, these animals have significantly reduced lung viral load post-challenge (FIG. 4) and more perivascular cuffing of lymphocytes with detectable CD4⁺ and CD8⁺ T cells, as well as B lymphocytes (FIG. 5, FIG. 9), again, suggesting that other immune mechanisms (such as T cells) may contribute to protection and should be considered in vaccine design against respiratory diseases such as COVID-19.

The data in these studies demonstrate that the SARS-CoV-2 combination nanovaccine platform induced high titer anti-S IgG antibody that persisted for extended time periods, anti-S IgG that bound to the RBD of multiple variants, and anti-S neutralizing antibody responses. Additionally, serum antigen-specific CD4⁺ and CD8⁺ T cell responses were detected following immunization with the nanovaccine that were associated with antigen-specific induction of multiple cytokines, including IFN-γ, IL-2, IL-4, and IL-17. An optimized formulation (i.e., 2× antigen dose nanovaccine) was identified that provided complete protection against lethal SARS-CoV-2 challenge demonstrating the promise of this platform as a next-generation SARS-CoV-2 vaccine.

### Example 2. Material and Methods

Polyanhydride nanoparticle synthesis and released protein characterization. Monomers of 1,8-bis(p-carboxyphenoxy)-3,6-dioxaoctane (CPTEG) and 1,6-bis(p-carboxyphenoxy) hexane (CPH) were synthesized as previously described (59, 60). Next, 20:80 CPTEG:CPH copolymer was synthesized via melt polycondensation for ~3 h (60). 1H nuclear magnetic resonance (1H NMR; DXR 500, Bruker, Billerica, MA) was used to determine polymer molecular weight, composition, and purity before proceeding to particle synthesis. Methods for making polyanhydride particles and compositions comprising polyanhydride particles are disclosed in U.S. Pat. Nos. 10,874,737; 8,927,024; and U.S. Patent Publication No. 2015/0216,888.

Polyanhydride particles encapsulating S, N, and CDNs (e.g., CdG) were synthesized via flash nanoprecipitation, similar to previous protocols (Ulery et al., PLoS One 6, e17642 (2011); Ross et al., Biomater Sci 7, 809-821 (2019)). Briefly, S and N proteins were purchased from Sino Biological Inc. (Beijing, China), dialyzed to nanopure water, and lyophilized overnight. Lyophilized CdG (cyclic di-GMP, sodium salt) was purchased from Cayman Chemical (Ann Arbor, MI). The 20:80 CPTEG:CPH polymer (with appropriate payload amounts) was dissolved 20 mg/mL in methylene chloride. The solution was sonicated for 30 s to ensure polymer dissolution and even distribution of the payload. Next, the solution was poured into chilled pentane (1:250 methylene chloride:pentane) and the resulting particles collected via vacuum filtration.

Nanoparticle size and morphology were verified with scanning electron microscopy (FEI Quanta 250, FEI, Hillsboro, OR) before use. The average size of the particles was 330-360 nm and is shown in FIG. 7. The pentablock copolymer micelles have a diameter of approximately 30 nm. Zeta potential analysis of polyanhydride nanoparticles (1 mg/mL), pentablock copolymer micelles (12 mg/mL), and mixture of nanoparticles and micelles was −47.9±3.2 mV, 4.3±0.3 mV, and 4.1±0.0 mV, respectively.

A representative sample of nanoparticles encapsulating S, N, and a co-adjuvant was placed in PBS at 37° C. and the total protein released was determined (FIG. 7). For protein release characterization, approximately 6 mg of 2% S, 2% N (w/w)-loaded 20:80 CPTEG:CPH nanoparticles was suspended in 200 μL of PBS, sonicated briefly, and placed on a shaker incubator at 37° C. 200 μL were removed and replaced with fresh PBS at the indicated time points, and protein release was measured using a micro bicinchoninic acid assay (Thermo Fisher Scientific, Waltham, MA). Following 17 days of release, the buffer was changed to 40 mM sodium hydroxide to accelerate the release of any remaining protein. The encapsulation efficiency of total protein (S and N) was calculated as the total amount of protein released divided by the theoretical amount of protein that would have been encapsulated during particle synthesis, which ranged between 8-20% in these studies.

SDS-PAGE gel analysis was performed using release supernatants from 1× antigen and 2× antigen nanovaccines. Sample concentrations were adjusted to 100 μg/mL, then diluted with an equal volume of 2× Laemmli Sample Buffer (Bio-Rad, catalog #161-0737), and 25 μL of each diluted sample was added to the respective wells of a polyacrylamide gel (4-20% MP TGX Gel 10 W 30 μL, Bio-Rad, catalog #4561093). The gel was subjected to 150 V for 60 min using 25 mM Tris, 192 mM glycine running buffer, placed in fixative solution (40% (v/v) ethanol, 10% (v/v) acetic acid) for 3 h, and then stained overnight using flamingo fluorescent gel stain (Bio-Rad, catalog #161-0491). The gel was imaged on an Invitrogen iBright™ FL1500 Imaging System the following morning. A representative SDS-PAGE of S protein upon release from the nanoparticles is shown in FIG. 7.

Pentablock copolymer micelle synthesis. Novel pentablock copolymer (PDEAEM-POE-POP-POE-PDEAEM) was synthesized by atom transfer radical polymerization as previously described (Adams et al., Macromol Chem Phys 214, 1321-1325 (2013); Determan et al., Polymer 46, 6933-6946 (2005). Briefly, prepared macroinitiator and DEAEM monomer synthesized the pentablock copolymer by utilizing copper (I) oxide nanoparticle catalysts and N-propylpyrilidine methanamine complexing ligands. The resulting pentablock copolymer was characterized with 1H NMR to determine purity and advanced polymer chromatography (Waters, Milford, MA) to determine molecular weight (Mₙ=13,292 g/mol; PDI=1.08) . . .

Animals. Female C₅₇BL/6 mice (6-8 week) were purchased from Charles River Laboratories (Wilmington, MA). Female transgenic K18-hACE2 (6-8 week) mice were purchased from The Jackson Laboratory (Bar Harbor, ME). All mice were housed under specific pathogen-free conditions where all bedding, caging, water, and feed were sterilized before use. Animal procedures were conducted with the approval of the Iowa State University Institutional Animal Care and Use Committee.

Immunizations. All animals received two subcutaneous immunizations at the nape of the neck delivered three weeks apart on days 0 and 21 post-prime vaccination (Table 2). Mice immunized with a 1× antigen dose received 500 μg of polyanhydride particles encapsulating 1 wt. % each S and N antigen, 5 mg of PBC micelles associated with 5 μg each S and N antigen, and 20 μg of CdG co-adjuvant; the CdG co-adjuvant was administered either as a soluble bolus (FIG. 1A) or with half of the CdG co-encapsulated in the polyanhydride nanoparticles with the S and N antigens (FIG. 1B-5). Mice receiving the 2× antigen dose received 500 μg of polyanhydride particles encapsulating 2 wt. % each S, N, and CdG co-delivered with 5 mg of PBC micelles associated with 10 μg each S, N, and CdG (FIGS. 3-5).

TABLE 2
Nanovaccine Formulations

| | | | | PBC Micelles (5 mg) | | |
|---|---|---|---|---|---|---|
| | Polyanhydride Nanoparticles (500 μg) | | | | | CdG |
| Nanovaccine | | | CdG | | | Co- |
| Formulation | S Antigen | N Antigen | Co-adjuvant | S Antigen | N Antigen | adjuvant |
| 1X Antigen | 5 μg | 5 μg | — | 5 μg | 5 μg | 20 μg |
| (soluble CdG) | | | | | | |
| 1X Antigen | 5 μg | 5 μg | 10 μg | 5 μg | 5 μg | 10 μg |
| 2X Antigen | 10 μg | 10 μg | 10 μg | 10 μg | 10 μg | 10 μg |

*All animals received two subcutaneous immunizations at the nape of the neck delivered three weeks apart on days 0 and 21 post-prime vaccination.

Antibody responses. Total IgG antibody titers were determined by enzyme-linked immunosorbent assay (ELISA). S protein or its receptor binding domain (RBD) variants were purchased from Sino Biological Inc. or Genscript Biotech Corp. (Piscataway, NJ), respectively. Four RBD variants were examined: wild-type (Wuhan-Hu-1-2019), Beta (B.1.351), B.1.617 (parent lineage of Delta and Kappa), and Omicron (B.1.1.529). Briefly, 100 μL/well of 0.5 μg/mL S or RBD was incubated overnight at 4° C. on high binding, 96-well plates. The plates were subsequently blocked with 300 μL/well 2% gelatin in PBS-T (PBS with 0.05% Tween 20) for 2 h at room temperature. Plates were washed 3× with PBS-T before adding 100 μL/well of 1% goat serum in PBS-T. Serum samples from mice were added at a 1:200 dilution and 1:2 serial dilutions were continued across the plate. Plates were incubated overnight at 4° C. After washing, plates were incubated 100 μL/well of 1:1000 alkaline phosphatase-conjugated anti-mouse IgG H&L (Jackson ImmunoResearch, West Grove, PA) for 2 h at room temperature. After a final wash, 100 μL/well of substrate buffer (50 mM sodium carbonate, 2 mM magnesium chloride, pH 9.3) containing 1 mg/mL p-nitrophenylphosphate was added to the plates. The optical density (405 nm) was measured after 30 min. Titer was considered the reciprocal of the last serum dilution with optical density above 0.2 (background).

Neutralizing antibody responses. The ability of serum antibodies to block interaction between the S protein RBD and ACE2 human cell surface receptor was determined using a surrogate virus neutralization test (sVNT) kit (GenScript Biotech Corp.). Serum samples were diluted 1:20 and the sVNT assay completed as per the manufacturer's instructions. This kit also provided positive and negative controls that were used in this assay.

Virus challenge. Wild-type SARS CoV-2 (NR-52281) isolated from a COVID-19 patient in Washington, USA, was acquired from BEI and propagated on Vero E6 cells (CRL-1586). Animals were anesthetized with intraperitoneal injection of ketamine/xylazine cocktail and infected intranasally with 2.5×10⁴ or 1×10⁵ PFU live SARS-CoV-2 virus. Body weight was measured daily, as well as morbidity and mortality monitored for two weeks. All activities were performed in an animal biosafety level 3 (ABSL-3) laboratory at Iowa State University with protocols approved by the Iowa State University Institutional Animal Care and Use Committee and Institutional Biosafety Committee.

Viral load. At five days post-challenge, four mice per treatment group were sacrificed to collect lung and brain tissue. The tissues were homogenized in sterile PBS using a Qiagen TissueLyser II. The homogenized tissues were centrifuged and supernatants were collected. Next, 100 μL of the supernatants were treated with 400 μL of Trizol. To that, 80 μL of chloroform was added and centrifuged at 12,000×g at 4° C. for 15 min. Isopropanol (200 μL) was added to the upper aqueous layer and centrifuged for 10 min at 12,000×g and 4° C. The RNA pellet was washed with ethanol and resuspended in 50 μL of nuclease-free water. The RNA concentration for each sample was normalized to 50 ng/μL and RT-qPCR was performed by using Luna Universal Probe One-Step RT-qPCR kit and primer-probe from IDT 2019-nCoV kit on the Bio-Rad CFX96 Real-Time system.

Histopathology and immunohistochemical (IHC) analyses. For histopathological analysis, lung tissue harvested at five days post-challenge was fixed with 10% buffered formalin. The tissues were processed in Excelsior AS tissue processor (Thermo Scientific) and embedded in paraffin with Tissue-Tek (Sakura Finetek). Histologic sections were deparaffinized in xylene and rehydrated through graded concentrations of ethanol. Sections were incubated in hematoxylin and eosin (H&E). For H&E staining, sections were incubated in freshly filtered Mayer's hematoxylin solution (BBC Biochemical, 3580) for 10 min and alcoholic eosin solution (American Mastertech, STE0457) for 2 min. Sections were differentiated individually in 0.05% lithium carbonate (Fisher, L119-500) and 70% ethanol, counterstained with 0.1% cresyl violet acetate solution (Sigma, C₅₀₄₂) for 6 min, and dehydrated through a graded ethanol series, cleared in xylene, cover slipped, and examined in a blinded manner by a board-certified veterinary pathologist (OF) using a BX-43 Olympus Trinocular brightfield microscope with an Olympus DP27 digital colored camera and CellSens imaging software (v1.15, Olympus Corporation).

For immunohistochemical (IHC) analysis, sections were deparaffinized and rehydrated using xylene, ethanol, and water washes. Sections were then treated with Reveal Decloaker (BioCare Medical, RV1000 at 1:10 dilution) under pressure for 45 min. For sections stained with anti-CD4 and anti-CD8a, endogenous peroxidase activity was first quenched via two 10 min H2O2 incubations. VECTASTAIN Universal Quick Kit (VectorLabs, PK-7800) was used to perform direct detection IHC. Sections were blocked with 2.5% normal horse serum from the kit for 20 min. Primary antibodies used were monoclonal biotinylated CD4/4SM95 (eBioscience, 13-9766-82) at 1:50 dilution and monoclonal biotinylated CD8a/4SM15 (eBioscience, 13-0808-82) at 1:50 dilution. Primary antibodies were diluted in Animal Free Blocker (SP5030) and incubated for 2 h. The kit streptavidin/peroxidase complex was applied for 5 min followed by treatment with ImmPACTNova Red peroxidase substrate solution (Vector Laboratories, SK4805) for 5 min, counterstained with hematoxylin for 5 min, dehydrated, and cover slipped. For sections stained with anti-CD20, endogenous peroxidase activity was quenched with FLX Peroxidase Blocking Reagent (BioCare Medical, IPB5000) for 5 min. Polyclonal CD20 (Invitrogen, PA5-16701) was diluted 1:100 in DaVinci Green Diluent (BioCare Medical, PD900) and incubated for 30 min followed by a 30 min incubation with Rb-on-Canine-HRP Polymer (BioCare Medical, RC542). Slides were then treated with DAB Chromogen Kit (BioCare Medical, IPK5010) for 5 min, counterstained with hematoxylin for 5 min, dehydrated, and cover slipped.

T cell assays. Mice were euthanized (n=8 mice/group) at 45 days post-immunization and axillary/brachial lymph nodes and spleen were harvested. Tissues were subsequently homogenized and filtered to a single cell suspension in complete DMEM media. Cells were diluted to a concentration of 5×10⁶ cells in 1 mL of PBS in 15 mL conical tubes, after which 1 mL of CFSE solution (2 μM CFSE in PBS) was added to the cells to bring the final concentration of CFSE to 1 μM. The cells were stained for 5 min at room temperature with constant gentle shaking. After 5 min, 0.5 mL of fetal bovine serum was added to the cell suspensions, mixed well, then 7.5 mL PBS was added to the tubes to bring to final volume of 10 mL. Cells were then centrifuged (300×g, 4° C.) for 10 min, resuspended in 5 mL cDMEM, and again centrifuged (300×g, 4° C.) for 10 min. Cells were then resuspended at 5×10⁶ cells/mL in cDMEM and 100 μL was added to the respective wells, resulting in 5×10⁵ cells/well. Following this, 100 μL of each stimulant was added to wells and allowed to stimulate cells for five days. For stimulants, S antigen and N antigen were both added to wells at a final concentration of 25 μg/mL.

Negative controls were pooled cells that received 100 μL cDMEM only. Positive controls were pooled cells that received polkweed mitogen at a final concentration of 2.5 μg/mL, and also a separate group of pooled cells that were pipetted in wells precoated with 1 μg/mL anti-CD3 monoclonal antibody overnight, and then received 5 μg/mL of anti-CD28 monoclonal antibody. An irrelevant antigen, hen-egg ovalbumin, was also added to separate wells containing pooled cells to ensure the response was antigen-specific. On days 2 and 3 post-stimulation, 20 μL of cDMEM was added to wells. After 5 days of stimulation, 100 μL of cell culture supernatants were collected and the levels of cytokine secretion were measured using the Bio-Rad BioPlex 200 system (Hercules, CA) for the following cytokines: IL-6, IL-1B, IL-17, TNF-α, IL-12, IL-10, IFN-γ, IL-5, IL-2, and IL-4. The remaining volume and cells were pipetted out of the plate and stained for flow cytometric analysis.

For flow cytometric analysis, cells were washed with PBS before being resuspended in 100 μL Zombie Aqua (BioLegend) at a 1:1000 dilution for 25 min, per manufacturer's instructions. After washing with 2 mL FACS buffer (1% fetal bovine serum, 0.1% sodium azide in phosphate buffered saline), Fc receptors were blocked to prevent non-specific antibody binding by incubating with 10 μL of 1 mg/mL rat IgG (Sigma Aldrich, St. Louis, MO) and 10 μg/mL of anti-CD16/32 (eBioscience) on ice. After 10 min of blocking, the cells were stained with 30 μL of FACs buffer containing a 1:100 dilution of fluorescently conjugated antibodies for CD4 (BioLegend, PerCp-Cy5.5, clone GK1.5) and CD8B (BioLegend, APC, clone YTS156.7.7) for 30 min on ice and in the dark. Following staining, cells were washed with FACS buffer and resuspended in 100 μL BD Stabilizing Fixative per manufacturer's instructions for flow cytometric analysis on a BD FACS Canto (BD Biosciences).

Statistical analyses. Statistical significance was determined using GraphPad Prism software (Version 9.3.1, GraphPad Software, LLC; La Jolla, CA). Significance in antibody titer, cell proliferation, or cytokine secretion (FIG. 1-2) was determined with a Mann-Whitney test. Optical density (FIGS. 3 and 6) and weight loss significance (FIG. 4) were determined via two-way ANOVA with Tukey's multiple comparisons. A log-rank Mantel Cox curve comparison determined survival significance (FIG. 4), while a one-way ANOVA with Tukey's multiple comparisons or Tukey t-test was used to determine significant neutralizing antibody or viral load differences based on the number of treatment groups (FIGS. 3 and 4).

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. An immunogenic composition comprising:
biodegradable particles comprising one or more polymers, wherein the polymers comprise monomers of one or more of 1,8-bis(p-carboxyphenoxy)-3,6-dioxaoctane (CPTEG), 1,6-bis(p-carboxyphenoxy) hexane (CPH), sebacic anhydride (SA), a SA homopolymer, a CPTEG:CPH copolymer, a CPTEG:SA copolymer, a CPH:SA copolymer, a CPH homopolymer, or a polyester copolymer;
at least one immunogenic protein of a sarbecovirus; and
at least one adjuvant;
wherein a first amount of the immunogenic protein and a first amount of the adjuvant are encapsulated within the biodegradable particles.

2. The immunogenic composition of claim 1, wherein the biodegradable particles comprise:
i) the CPTEG:CPH copolymer in a molar ratio of about 20:80;
ii) the CPTEG:SA copolymer in a molar ratio of 10:90;
iii) the CPH:SA copolymer in a molar ratio of 20:80; or
iv) the polyester copolymer comprises PLGA in a molar ratio of 50:50.

3. The immunogenic composition of claim 1, further comprising pentablock copolymer micelles, wherein a second amount of the immunogenic protein, a second amount of the adjuvant, or both are entrapped within one or more of the pentablock copolymer micelles.

4. The immunogenic composition of claim 3, wherein the pentablock copolymer micelle comprises poly((diethylamino)-ethyl methacrylate) (PDEAEM), a polyolefin elastomer (POE), and a polyolefin plastomer (POP).

5. The immunogenic composition of claim 4, wherein the pentablock copolymer micelle comprises PDEAEM and a POE, wherein the POE is poloxamer 407.

6. The immunogenic composition of claim 3, wherein the composition comprises about 0.5 milligrams to about 100 milligrams of the pentablock copolymer micelles.

7. The immunogenic composition of claim 3, wherein immunogenic protein and the adjuvant are present in the pentablock copolymer micelles in an amount of about 0.1 micrograms to about 500 micrograms each.

8. The immunogenic composition of claim 1, wherein the adjuvant is a Stimulator of Interferon Genes (STING) activating adjuvant, a Toll-like receptor (TLR) agonist, a nucleotide oligomerization domain (NOD)-like receptor (NLR) agonist, a RIG-like receptor (RLR), a liposome, an aluminum salt, a mineral salt, an oil emulsion, a polymer, a polysaccharide, a saponin, or a CpG polynucleotide.

9. The immunogenic composition of claim 8, wherein a STING activating adjuvant is present in the composition and the STING activating adjuvant comprises a cyclic dinucleotide selected from the group consisting of cyclic di-guanosine monophosphate (c-di-GMP), cyclic di-adenosine monophosphate (c-di-AMP), and a cyclic guanosine monophosphate-adenosine monophosphate (cGAMP).

10. The immunogenic composition of claim 1, wherein the composition comprises about 5 micrograms to about 1000 milligrams of the biodegradable particles.

11. The immunogenic composition of claim 1, wherein the immunogenic protein and the adjuvant are present in the biodegradable particles in an amount of about 0.1 micrograms to about 500 micrograms each.

12. The immunogenic composition of claim 1, wherein the sarbecovirus is severe acute respiratory syndrome coronavirus (SARS-CoV), severe acute respiratory syndrome coronavirus 2 (SARS-CoV-2), Middle East respiratory syndrome coronavirus (MERS-CoV), or a variant thereof.

13. The immunogenic composition of claim 1, wherein the immunogenic protein comprises one or more of a spike protein (S protein), a nucleocapsid protein (N protein), an envelope protein (E protein), and a membrane protein (M protein).

14. The immunogenic composition of claim 1, wherein the immunogenic protein comprises a sarbecovirus spike protein (S protein) and a sarbecovirus nucleocapsid protein (N protein).

15. An immunogenic composition comprising:
biodegradable polyanhydride particles comprising 1,8-bis(p-carboxyphenoxy)-3,6-dioxaoctane (CPTEG) and 1,6-bis(p-carboxyphenoxy) hexane (CPH) copolymers in a molar ratio of about 20:80;
pentablock copolymer micelles comprising poloxamer 407 and poly((diethylamino)-ethyl methacrylate) (PDEAEM);
a spike protein (S protein) and a nucleocapsid protein (N protein) of SARS-CoV-2 virus; and
an adjuvant comprising cyclic di-guanosine monophosphate (c-di-GMP);
wherein a first amount of the S protein, a first amount of the N protein, and a first amount of the adjuvant are encapsulated within the biodegradable particles; and
a second amount of the S protein, a second amount of the N protein, and a second amount of the adjuvant are entrapped within the pentablock copolymer micelles.

16. The immunogenic composition of claim 15, wherein the first amount of the S protein, the first amount of the N protein, the second amount of the S protein, and the second amount of the N protein, are present in an amount of about 0.1 micrograms to about 500 micrograms each; and
the first amount of the adjuvant and the second amount of the adjuvant are present in an amount of about 0.1 micrograms to about 500 micrograms each.

17. A method of inducing an immune response against a sarbecovirus in a subject comprising administering to the subject an effective amount of the immunogenic composition of claim 1, thereby inducing an immune response.

18. The method of claim 17, wherein the immune response comprises both a local immune response and systemic protective immune response to sarbecovirus.

19. The method of claim 17, wherein the administering step comprises one or more of intranasal administration and parenteral administration.


SEQUENCE LISTING (4 sequences)

SEQ ID NO: 1 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 419 aa, Severe acute respiratory syndrome-related coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - protein, 222 aa, Severe acute respiratory syndrome-related coronavirus
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 4 - protein, 75 aa, Severe acute respiratory syndrome-related coronavirus
MYSFVSEETGTLIVNSVLLFLAFVVFLLVTLAILTALRLCAYCCNIVNVSLVKPSFYVYSRVKNLNSSRVPDLLV